(19) United States
(12) Patent Application Publication
(10) Pub. No.: US 2009/0298061 A1
(43) Pub. Date: Dec. 3, 2009

(54) Diagnostic Methods for the Prediction of Therapeutic Success, Recurrence Free and Overall Survival in Cancer Therapy

(75) Inventor: Ralph Markus Wirtz, Koln (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Healthcare Diagnostics Inc.

(21) Appl. No.: 11/989,830

(22) PCT Filed: Jul. 20, 2006
(86) PCT No.: PCT/US2006/028180

§ 371 Date: Jul. 28, 2009

Related U.S. Application Data
(60) Provisional application No. 60/704,207, filed on Jul. 29, 2005.
(60) Provisional application No. 60/715,407, filed on Sep. 9, 2005.

(51) Int. Cl. C12Q 1/68

(52) U.S. Cl. 435/6

(57) ABSTRACT

Described are 12 human genes which are differentially expressed in neoplastic tissues of patients responding well to treatment as compared to patients not responding well as determined by overall survival time in the non responding cohort. Moreover, methods for prognosis of the therapeutic success in cancer therapy are described. These methods are based on determination of expression levels of particular genes which are differentially expressed in cancer patients, preferably the genes encoding VEGFC, ERBB3 and Her2/neu, prior to the onset of anti-cancer chemotherapy. These methods are particularly useful in the investigation of advanced head and neck cancer, but are useful in the investigation of other types of cancer and therapies as well.

The present invention relates to 12 human genes, which are differentially expressed in neoplastic tissue of patients responding well, to treatment as compared to patients not responding well as determined by overall survival time. Thus, the present invention relates to methods for prognosis of the therapeutic success of cancer therapy. In a preferred embodiment of the invention it relates to methods for predicting therapeutic success of combinations of signal transduction inhibitors, therapeutic antibodies, radiotherapy and/or chemotherapy. The methods of the invention are based on the determination of the expression level of particular genes which are differentially expressed in cancer patients, preferably the genes encoding VEGFC, ERBB3 and Her2/neu, prior to the onset of anti-cancer chemotherapy. The methods of the invention are particularly useful in the investigation of advanced head and neck cancer, but are useful in the investigation of other types of cancer as well, including lung, ovarian, cervix, stomach, pancreas, prostate, head and neck, renal cell, colon and breast cancer. Of particular interest are head and neck, renal cell, colon and breast cancer.

Cancer is the second leading cause of death in the United States after cardiovascular disease. One in three Americans will develop cancer in his or her lifetime, and one of every four Americans will die of cancer. Tumors in general are classified based on different parameters, such as tumor size, invasion status, involvement of lymph nodes, metastasis, histopathology, immunohistochemical markers, and molecular markers (WHO; International Classification of diseases, 10^{th} edition (ICD-10), WHO; Sabin and Wittekind (eds): TNM Classification of Malignant Tumors, Wiley, New York (1997)). With the recent advances in gene chip technology, researchers are increasingly focusing on the categorization of tumors based on the distinct expression of marker genes Sorlie et al., PNAS USA 98(19) (2001), 10869-74; van't Veer et al., Nature 415 (6871) (2002), 530-6.

It is a well established fact, that adjuvant systemic treatment after surgery reduces the risk of disease relapse and death in patients with primary operable cancer. In general, all patients of a given cohort do receive the same treatment, even though many will fail in treatment success. Bio-markers predicting tumor response can function as sensitive short-term surrogates of long-term outcome. The use of such bio-markers will make chemotherapy more effective for the individual patient and will allow changing regimen early in the case of non-responding tumors. Although much effort has been devoted in developing an optimal clinical treatment course for individual patients with cancer, only little progress has been made in predicting the individual's response to a certain therapy.

Tumors of the head and neck, which include the upper aerodigestive tract (oral cavity, oropharynx, hypopharynx, and larynx), account for over 40,000 cases of cancer per year in the US. The most common histology of head and neck tumor is squamous cell carcinoma. The main prognostic variables of head and neck squamous cell carcinoma (HNSCC) are the location and size of the tumor, the presence of distant metastasis, and the presence of cervical lymph node (LN) metastasis. About 40%-50% of patients with advanced disease (Stage III and IV) recur, and approximately 80% of recurrences occur within the first two years. Most of the clinical decisions regarding therapy are commonly based upon clinical staging, which relies on nodal status and tumor size. No biomarkers analogous to the estrogen receptor or HER2 in breast cancer, or c-KIT in gastrointestinal tumors, exist for HNSCC patients, suggesting that genomic profiling studies may be useful for identifying new biomarkers with prognostic or predictive value. The prognostication of HNSCC is largely based upon the tumor size and location and the presence of lymph node metastases. Despite the aggressive multimodality treatment of HNSCC patients with surgery, chemotherapy, and radiation therapy, approximately 40%-50% of patients with advanced disease recur. To date, there are no reliable biomarkers to predict who will have poor clinical outcome and should receive more intense or targeted regimens.

Gene expression profiling has been used to identify subclasses of HNSCC tumors. However head and neck squamous cell carcinomas show significant heterogeneity. Therefore their clinical behaviour so far could not be predicted using the current set of clinical markers. Previous microarray-based studies of HNSCC have primarily focused on tumor versus normal patterns of expression (El-Naggar et al., Oncogene 21 (2002), 8206-19; Hwang et al., Oral Oncol. 39 (2003), 259-68 and Leethanakul et al., Oral Oncol. 39 (2003), 248-58. Others have suggested that there might be subtypes of HNSCC (Belbin et al., Cancer Res. 62 (2002), 1184-90). However, to date no study has shown statistically significant differences in clinical outcomes between subtypes of HNSCC based upon gene expression patterns. Chung et al. (Cancer Cell 5 (2004), 489-500) identified four distinct subtypes of HNSCC based upon an “intrinsic analysis” and showed that these subtypes had differences in recurrence-free survival and overall survival. However, the number of genes they used to subclassify the tumors is enormous (582 cDNA clones). These expression signatures were revealing for the highly complex biology that underlies HNSCC and suggests that further analysis also by functional assays is needed.

It is well known that the epidermal growth factor receptor (EGFR) pathway is important for HNSCC. The gene set presented by Chung et al. (2004) contained at least three genes from this pathway, including TGFα, FGF-BP, and MMK6. TGFα is a ligand for EGFR and a critical activator of the EGFR pathway in HNSCC. FGF-BP is a promoter of angiogenesis, induced by EGF in vitro and by the ectopic expression of MMK6, which is a MAP kinase downstream of the EGFR. Among the 60 tumors that were analyzed by microarray, 56 were also analyzed by immunohistochemistry (IHC) for the presence of EGFR and for the Tyr-1173 phosphorylated form of EGFR. Of these 56 tumors, 54 were positive for EGFR expression and 35/54 of the EGFR-expressing tumors were also positive for P-Tyr-1173-EGFR. Among the Group 1 tumors, all tested were IHC positive for EGFR and a high percentage ( 15/19, 79%) were positive for P-Tyr-1173-EGFR (50% of Group 2, 75% of Group 3, and 38% of Group 4 tumors were positive for P-Tyr-1173-EGFR). These data suggest, that EGFR signaling is typically active in Group 1 and 3 tumors. These data also indicate, that not all EGFR+tumors have an activated EGFR, which is likely influenced by the presence of ligands like TGFα. However, based on these data no clear distinction between EGFR positive tumors can be drawn. Also no evidence of a knowledge based approach can be deduced, that would analyze the correlation between the presence of other EGFR family dimerization partners and their effect on survival. This is particularly striking, as they subdivide the “EGFR subclass” present in group 1 and 3 tumors in different groups when correlated with clinical outcome. Moreover, Chung et al. (2004) developed predictors for clinical parameters, utilizing two different supervised statistical analyses. Their predictors included (1) a simple gene selection method coupled with sample predictions made using an Euclidian correlation to the K-Nearest Neighbors (KNN) of a given sample (K=3) and (2) PAM analysis as described by Tibshirani et al., PNAS USA 99 (2002), 6567-72). However, the authors obtained prediction accuracy of as little as 60% (KNN) and 58% (PAM) when performing a 10-fold cross validation analysis and by using at least 50 to up to 200 genes. In summary the existing state of the art technology impressively underlines the inability to predict clinical outcome of head and neck cancer even when performing genome wide analysis and using statistical methods.

Breast cancer claims the lives of approximately 40,000 women and is diagnosed in approximately 200,000 women annually in the United States alone. For breast cancer, predictions are usually based on standard clinical parameters such as tumor stage and grade, estrogen (ER) and progesterone (PgR) receptor status, growth rate, and over-expression of the HER2/neu and p53 oncogenes. However, evidences about the association of ER and/or PgR gene expression with outcome prediction for adjuvant endocrine chemotherapy is still controversial. A number of studies have shown that levels of ER and PgR gene expression in breast cancer patients are of prognostic importance, independently from the administration of subsequent adjuvant chemotherapy. From the theoretical point of view, it is quite unexpected that the therapeutic response of patients with breast cancer might be independent from the ER/PgR status. It is more probable that the prognostic impact of the expression of the ER/PgR depends on other parameters, for example the ERBB2 receptor. However, studying such factors using conventional biological techniques is problematic, since all these analyses survey one gene at a time.

Researchers are increasingly focusing on the categorization of tumors based on distinct expression of marker genes. In this respect DNA microarray technology has been very useful for quantitative measurements of expression levels of thousands of genes simultaneously in one sample. So far this technology has been applied for the classification of cancer tissues and the prediction of metastasis, patient's outcome and tumor response to chemotherapy.

Nevertheless, chemotherapy remains a mainstay in therapeutic regimens offered to patients with breast cancer, particularly those who have cancer that has metastasized from its site of origin. There are several chemotherapeutic agents that have demonstrated activity in the treatment of cancer and research is continuously in an attempt to determine optimal drugs and regimens. However, different patients tend to respond differently to the same therapeutic regimens. Currently, the individual response to certain therapy can only be assessed statistically, based on data of clinical studies. There is still a great number of patients who do not benefit from systemic chemotherapy. Most types of cancer are very heterogeneous in their aggressiveness and treatment response. They contain different genetic mutations and variations affecting growth characteristics and sensitivity to drugs. Identification of each tumor's molecular fingerprint, therefore, could help segregate patients who have particularly aggressive tumors or who need to be treated with specific beneficial therapies. As research involving genetics and associated responses to treatment matures, standard treatment will undoubtedly become more individualized, enabling physicians to provide specific treatment regimens matched with a tumor's genetic profile to ensure optimal outcome. As an alternative therapeutic concept neo-adjuvant or primary systemic therapy (PST) can be offered to those patients with larger inoperable breast cancers. The PST in general does not offer a survival advantage over standard adjuvant treatment, but may identify patients with a pathologically—confirmed complete response (CR). In this therapeutic setting such biomarkers capable of predicting response can be measured in vivo by correlating gene expression directly with the tumor response.

Thus, the technical problem underlying the present invention is to provide biological markers allowing to determine cancer status, preferably HNSCC, breast and colon cancer, and to predict therapeutic success of a given treatment regiment.

The solution to said technical problem is achieved by providing the embodiments characterized in the claims. The present invention is based on the unexpected finding, that particular human genes (listed in Table 1) are differentially expressed in neoplastic tissues of patients having bad prognosis due to lack of sustained response to anti-cancer regimens, as compared to patients having better outcome due to sustained response to therapy. Moreover by a knowledge based approach an underlying biological process could be identified that dramatically affects the overall survival of head and neck cancer patients, irrespectively of the administered standard therapeutic regimen. The early recruitment of lymphatic vessels is of major importance for the overall survival of patients with advanced head and neck cancer. In particular, the expression of the growth factor receptor ligand VEGFC and its high affinity receptor FLT-4 does correlate with dramatically worsened prognosis. Therefore, therapeutic interventions targeting these activities are most probably advantageous for the treatment of head and neck cancer. Surprisingly, the presence of certain EGFR-family members (ERBB2//Her-2/neu, ERBB3 and ERBB4) did account for less aggressive tumors and has high prognostic/predictive value. Target genes for newly available therapeutics (Iressa, sorafenib, SU 11248, Trastuzumab, Avastin), i.e. EGFR and VEGF alpha, were almost equally expressed in good and bad outcome patients, and therefore could be administered to almost all patients. However, especially for the bad prognosis patients, a benefit from such therapeutic strategies could be apparent, as the standard chemotherapy regimens fail in these situations. Similar processes could be identified in breast and colon cancer patients. Therefore this invention comprises also the prediction and prognosis of breast and colon cancer based on said genes as described in Table 1.

A further important part of the present invention is the identification of a biological motif, i.e. the recruitment of lymphatic vessels by the expression of VEGFC and the subsequent interaction with the FLT-4 cell surface receptors on endothelial cells, as being of major importance for the overall survival of cancer patients. This new concept is especially fruitful for respective anti-cancer strategies by using anti-VEGFC antibodies, VEGFC mimetic ligands and inhibitors of FLT-4 receptors, such as small molecules or antibodies.

Response to a local and systemic therapy may be the prolonged recurrence free survival time after intervention for the primary tumor, but may also reflect the over all survival time. Hence, elevated or decreased levels of expression in one or several of the marker genes of Table 1 at the time of tumor surgery or prior to any intervention (e.g. biopsy sample) was found to provide valuable information on whether or not a patient is likely to progress despite a given mode of therapy. This would also imply, that those individuals predicted not to progress within a given time frame (e.g. 5 years) will benefit from such chemotherapy regimen and that their tumors will respond to chemotherapy. In a preferred embodiment of the invention, said given mode of chemotherapy is targeted therapy such as small molecule inhibitors (e.g. Iressa, Sorafenib), and/or therapeutic antibodies (e.g. Trastuzumab, Bevacizumab) directed to the genes being identified as prognostic/predictive markers.

Thus, the present invention relates to a method for predicting therapeutic success of a given mode of treatment in a patient having cancer or for adapting the therapeutic regimen based on individualized risk assessment for a patient having cancer, comprising

(a) obtaining a biological sample from said patient;
(b) determining the pattern of expression levels of at least one marker gene of the group of marker genes listed in Table 1;
(c) comparing the pattern of expression levels determined in (b) with one or several reference pattern(s) of expression levels; and
(d) predicting therapeutic success of a given mode of treatment in said subject or implementing therapeutic regimen targeting said marker genes in said subject based on the outcome of the comparison in step (c).

“Differential expression”, or “expression” as used herein, refers to both quantitative as well as qualitative differences in the genes expression patterns observed in at least two different individuals or samples taken from individuals. Differential expression may depend on differential development, different genetic background of tumor cells and/or reaction to the tissue environment of the tumor. Differentially expressed genes may represent “marker genes,” and/or “target genes”. The expression pattern of a differentially expressed gene disclosed herein may be utilized as part of a prognostic or diagnostic cancer evaluation.

The term “pattern of expression levels” refers, e.g., to a determined level of gene expression compared either to a reference gene (e.g. housekeeper) or to a computed average expression value (e.g. in DNA-chip analyses). A pattern is not limited to the comparison of two genes but is more related to multiple comparisons of genes to reference genes or samples. A certain “pattern of expression levels” may also result and be determined by comparison and measurement of several genes disclosed hereafter and display the relative abundance of these transcripts to each other.

Alternatively, a differentially expressed gene disclosed herein may be used in methods for identifying reagents and compounds and uses of these reagents and compounds for the treatment of cancer as well as methods of treatment. The differential regulation of the gene is not limited to a specific cancer cell type or clone, but rather displays the interplay of cancer cells, muscle cells, stromal cells, connective tissue cells, other epithelial cells, endothelial cells of blood vessels as well as cells of the immune system (e.g. lymphocytes, macrophages, killer cells).

A “reference pattern of expression levels”, within the meaning of the invention shall be understood as being any pattern of expression levels that can be used for the comparison to another pattern of expression levels. In a preferred embodiment of the invention, a reference pattern of expression levels is, e.g., an average pattern of expression levels observed in a group of healthy or diseased individuals, serving as a reference group.

“Primer pairs” and “probes”, within the meaning of the invention, shall have the ordinary meaning of this term which is well known to the person skilled in the art of molecular biology. In a preferred embodiment of the invention “primer pairs” and “probes”, shall be understood as being polynucleotide molecules having a sequence identical, complementary, homologous, or homologous to the complement of regions of a target polynucleotide which is to be detected or quantified.

“Individually labeled probes”, within the meaning of the invention, shall be understood as being molecular probes comprising a polynucleotide or oligonucleotide and a label, helpful in the detection or quantification of the probe. Preferred labels are fluorescent labels, luminescent labels, radioactive labels and dyes.

“Arrayed probes”, within the meaning of the invention, shall be understood as being a collection of immobilized probes, preferably in an orderly arrangement. In a preferred embodiment of the invention, the individual “arrayed probes” can be identified by their respective position on the solid support, e.g., on a “chip”.

The phrase “tumor response”, “therapeutic success”, or “response to therapy” refers, in the adjuvant chemotherapeutic setting to the observation of a defined tumor free or recurrence free survival time (e.g. 2 years, 4 years, 5 years, 10 years). This time period of disease free survival may vary among the different tumor entities but is sufficiently longer than the average time period in which most of the recurrences appear. In a neo-adjuvant therapy modality, response may be monitored by measurement of tumor shrinkage due to apoptosis and necrosis of the tumor mass.

The term “recurrence” or “recurrent disease” includes distant metastasis that can appear even many years after the initial diagnosis and therapy of a tumor, or local events such as infiltration of tumor cells into regional lymph nodes, or occurrence of tumor cells at the same site and organ of origin within an appropriate time.

“Prediction of recurrence” or “prediction of therapeutic success” does refer to the methods described in this invention. Wherein a tumor specimen is analyzed for it's gene expression and furthermore classified based on correlation of the expression pattern to known ones from reference samples. This classification may either result in the statement that such given tumor will develop recurrence and therefore is considered as a “non responding” tumor to the given therapy, or may result in a classification as a tumor with a prolonged disease free post therapy time.

“Biological activity” or “bioactivity” or “activity” or “biological function”, which are used interchangeably, herein mean an effector or antigenic function that is directly or indirectly exerted by a polypeptide (whether in its native or denatured conformation), or by any fragment thereof in vivo or in vitro. Biological activities include but are not limited to binding to polypeptides, binding to other proteins or molecules, enzymatic activity, signal transduction, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “marker” or “biomarker” refers to a biological molecule, e.g., a nucleic acid, peptide, hormone, etc., whose presence or concentration can be detected and correlated with a known condition, such as a disease state.

The term “marker gene,” as used herein, refers to a differentially expressed gene whose expression pattern may be utilized as part of a predictive, prognostic or diagnostic process in malignant neoplasia or cancer evaluation, or which, alternatively, may be used in methods for identifying compounds useful for the treatment or prevention of malignant neoplasia and head and neck, colon or breast cancer in particular. A marker gene may also have the characteristics of a target gene.

“Target gene”, as used herein, refers to a differentially expressed gene involved in cancer, e.g., head and neck, colon or breast cancer in a manner in which modulation of the level of the target gene expression or of the target gene product activity may act to ameliorate symptoms of malignant neoplasia and head and neck, colon or breast cancer in particular. A target gene may also have the characteristics of a marker gene.

The term “neoplastic lesion” or “neoplastic disease” or “neoplasia” refers to a cancerous tissue this includes carcinomas, (e.g., carcinoma in situ, invasive carcinoma, metastatic carcinoma) and pre-malignant conditions, neomorphic changes independent of their histological origin (e.g. ductal, lobular, medullary, mixed origin). The term “cancer” as used herein includes carcinomas, (e.g., carcinoma in situ, invasive carcinoma, metastatic carcinoma) and pre-malignant conditions, neomorphic changes independent of their histological origin. The term “cancer” is not limited to any stage, grade, histomorphological feature, invasiveness, agressivity or malignancy of an affected tissue or cell aggregation. In particular stage 0 cancer, stage I cancer, stage II cancer, stage III cancer, stage IV cancer, grade I cancer, grade II cancer, grade III cancer, malignant cancer, primary carcinomas, and all other types of cancers, malignancies and transformations associated with the head and neck, colon or breast cancer are included. The terms “neoplastic lesion” or “neoplastic disease” or “neoplasia” or “cancer” are not limited to any tissue or cell type they also include primary, secondary or metastatic lesions of cancer patients, and also comprises lymph nodes affected by cancer cells or minimal residual disease cells either locally deposited (e.g. bone marrow, liver, kidney) or freely floating throughout the patients body.

Furthermore, the term “characterizing the state of a neoplastic disease” is related to, but not limited to, measurements and assessment of one or more of the following conditions: Type of tumor, histomorphological appearance, dependence on external signal (e.g. hormones, growth factors), invasiveness, motility, state by TNM (2) or similar, agressivity, malignancy, metastatic potential, and responsiveness to a given therapy.

The terms “biological sample” or “clinical sample”, as used herein, refer to a sample obtained from a patient. The sample may be of any biological tissue or fluid. Such samples include, but are not limited to, sputum, blood, blood cells (e.g., white cells), tissue or fine needle biopsy samples, cell-containing body fluids, free floating nucleic acids, urine, peritoneal fluid, and pleural fluid, or cells there from. Biological samples may also include sections of tissues such as frozen or fixed sections taken for histological purposes. A biological sample to be analyzed is tissue material from neoplastic lesion taken by aspiration or punctuation, excision or by any other surgical method leading to biopsy or resected cellular material. Such biological sample may comprise cells obtained from a patient. The cells may be found in a cell “smear” collected, for example, by a nipple aspiration, ductal lavarge, fine needle biopsy or from provoked or spontaneous nipple discharge. In another embodiment, the sample is a body fluid. Such fluids include, for example, blood fluids, lymph, ascitic fluids, gynecological fluids, or urine but not limited to these fluids.

The term “therapy modality”, “therapy mode”, “regimen” or “chemo regimen” as well as “therapy regimen” refers to a timely sequential or simultaneous administration of anti-tumor, and/or immune stimulating, and/or blood cell proliferative agents, and/or radiation therapy, and/or hyperthermia, and/or hypothermia for cancer therapy. The administration of these can be performed in an adjuvant and/or neoadjuvant mode. The composition of such “protocol” may vary in the dose of the single agent, timeframe of application and frequency of administration within a defined therapy window. Currently various combinations of various drugs and/or physical methods, and various schedules are under investigation.

By “array” or “matrix” is meant an arrangement of addressable locations or “addresses” on a device. The locations can be arranged in two dimensional arrays, three dimensional arrays, or other matrix formats. The number of locations can range from several to at least hundreds of thousands. Most importantly, each location represents a totally independent reaction site. Arrays include but are not limited to nucleic acid arrays, protein arrays and antibody arrays. A “nucleic acid array” refers to an array containing nucleic acid probes, such as oligonucleotides, polynucleotides or larger portions of genes. The nucleic acid on the array is preferably single stranded. Arrays wherein the probes are oligonucleotides are referred to as “oligonucleotide arrays” or “oligonucleotide chips.” A “microarray,” herein also refers to a “biochip” or “biological chip”, an array of regions having a density of discrete regions of at least about 100/cm², and preferably at least about 1000/cm². The regions in a microarray have typical dimensions, e.g., diameters, in the range of between about 10-250 μm, and are separated from other regions in the array by about the same distance. A “protein array” refers to an array containing polypeptide probes or protein probes which can be in native form or denatured. An “antibody array” refers to an array containing antibodies which include but are not limited to monoclonal antibodies (e.g. from a mouse), chimeric antibodies, humanized antibodies or phage antibodies and single chain antibodies as well as fragments from antibodies.

“Small molecule” as used herein, is meant to refer to a compound which has a molecular weight of less than about 5 kD and most preferably less than about 4 kD. Small molecules can be nucleic acids, peptides, polypeptides, peptidomimetics, carbohydrates, lipids or other organic (carbon-containing) or inorganic molecules. Many pharmaceutical companies have extensive libraries of chemical and/or biological mixtures, often fungal, bacterial, or algal extracts, which can be screened with any of the assays of the invention to identify compounds that modulate a bioactivity.

The terms “modulated” or “modulation” or “regulated” or “regulation” and “differentially regulated” as used herein refer to both upregulation [i.e., activation or stimulation (e.g., by agonizing or potentiating] and down regulation [i.e., inhibition or suppression (e.g., by antagonizing, decreasing or inhibiting)].

“Transcriptional regulatory unit” refers to DNA sequences, such as initiation signals, enhancers, and promoters, which induce or control transcription of protein coding sequences with which they are operably linked. In preferred embodiments, transcription of one of the genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally occurring forms of the polypeptide.

The term “derivative” refers to the chemical modification of a polypeptide sequence, or a polynucleotide sequence. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived. The term “derivative” furthermore refers to phosphorylated forms of a polypeptide sequence or protein.

“CANCER GENES” or “CANCER GENE” as used herein refers to the polynucleotides disclosed in Table 1, as well as derivatives, fragments, analogs and homologues thereof, the polypeptides encoded thereby as well as derivatives, fragments, analogs and homologues thereof and the corresponding genomic transcription units which can be derived or identified with standard techniques well known in the art using the information disclosed in Tables 1 to 4. The Gene symbol, Gene Description, Reference, Locus link ID, Unigene ID, and OMIM number are shown in Table 1.

A “CANCER GENE” polynucleotide can be single- or double-stranded and comprises a coding sequence or the complement of a coding sequence for a “CANCER GENE” polypeptide. Degenerate nucleotide sequences encoding human “CANCER GENE” polypeptides, as well as homologous nucleotide sequences which are at least about 50, 55, 60, 65, 70, preferably about 75, 90, 96, or 98% identical to the nucleotide sequences of Table 1 also are “CANCER GENE” polynucleotides.

“CANCER GENE” polypeptides according to the invention comprise a polypeptide of Table 1 or derivatives, fragments, analogues and homologues thereof. A “CANCER GENE” polypeptide of the invention therefore can be a portion, a full-length, or a fusion protein comprising all or a portion of a “CANCER GENE” polypeptide.

“CANCER GENE” polypeptide variants which are biologically active, i.e., retain a “CANCER GENE” activity, can be also regarded as “CANCER GENE” polypeptides. Preferably, naturally or non-naturally occurring “CANCER GENE” polypeptide variants have amino acid sequences which are at least about 60, 65, or 70, preferably about 75, 80, 85, 90, 92, 94, 96, or 98% identical to any of the amino acid sequences of the polypeptides encoded by the genes in Table 1 or the polypeptides encoded by any of the polynucleotides of Table 1 or a fragment thereof.

Variations in percent identity can be due, for example, to amino acid substitutions, insertions, or deletions. Amino acid substitutions are defined as one for one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine.

Amino acid insertions or deletions are changes to or within an amino acid sequence. They typically fall in the range of about 1 to 5 amino acids. Guidance in determining which amino acid residues can be substituted, inserted, or deleted without abolishing biological or immunological activity of a “CANCER GENE” polypeptide can be found using computer programs well known in the art, such as DNASTAR software. Whether an amino acid change results in a biologically active “CANCER GENE” polypeptide can readily be determined by assaying for “CANCER GENE” activity, as described for example, in the specific Examples, below. Larger insertions or deletions can also be caused by alternative splicing. Protein domains can be inserted or deleted without altering the main activity of the protein.

The prediction of therapeutic success or the investigation of the response to a treatment can be performed immediately after surgery or at time of first biopsy, at a stage in which other methods can not provide the required information on the patient's response to chemotherapy. Hence the current invention also provides means to decide—shortly after tumor surgery—whether or not a certain mode of chemotherapy is likely to be beneficial to the patient's health and/or whether to maintain or change the applied mode of chemotherapy treatment.

The different expression levels of the genes of the present invention is not limited to a specific cancer or neoplastic lesion in a certain tissue of the human body. Genes undergoing expressional changes as a response to a chemotherapeutic agent, can serve further on as monitoring markers for the therapy and, if they do correlate with the clinical outcome, such genes may also work as efficacy biomarkers.

In a preferred embodiment of the methods of the present invention the cancer is Head and Neck Cancer. However this invention also relates to predictive/prognostic value of said genes in colorectal and breast cancer.

The methods of the present invention comprise comparing the level of mRNA expression of a single or plurality (e.g. 1, 2, 3, 4, 5 or 12) of marker genes listed in Table 1 in a patient sample, and the average level of expression of the marker gene(s) in a sample from a control subject (e.g., a human subject without cancer). Comparison of the (pattern of) expression levels of one or several marker genes can also be performed on any other reference (e.g. tissue samples from responding tumors).

The methods of the present invention also comprise comparing the (pattern of) expression levels of mRNA of a single or plurality (e.g. 1, 2, 3, 4, 5 or 12) of marker genes in an unclassified patient sample, and the (pattern of) expression levels of the marker gene(s) in a sample cohort comprising patients responding in different intensity to an administered adjuvant cancer therapy. In a preferred embodiment of this invention the specific expression of the marker genes can be utilized for discrimination of responders and non-responders to a targeted or chemotherapeutic intervention.

The control level of mRNA expression (or the reference pattern(s) of expression levels) is the average level of expression of the marker gene(s) in samples from several (e.g., 2, 4, 8, 10, 15, 30 or 50) control subjects. These control subjects may also be affected by cancer and be classified by their clinical and not necessarily by their individual expression profile.

As elaborated below, a significant change in the level of expression of one or more of the marker genes in the patient sample relative to the control (or reference) level provides significant information regarding the patient's cancer status and responsiveness to chemotherapy, preferably targeted chemotherapy. In the method of the present invention the marker genes listed in Table 1 may also be used in combination with well known cancer marker genes (e.g. Ki-67, p53 and PTEN).

According to the invention, the marker genes are selected such that the positive predictive value of the methods of the invention is at least about 10%, preferably about 25%, more preferably about 50% and most preferably about 90% in any of the following conditions: stage 0 cancer patients, stage I cancer patients, stage II cancer patients, stage III cancer patients, stage IV cancer patients, grade I cancer patients, grade II cancer patients, grade III cancer patients, malignant cancer patients, patients with primary carcinomas, and all other types of cancers, malignancies and transformations associated with the head and neck, colon and breast.

The detection of marker gene expression is not limited to the detection within a primary, secondary or metastatic lesion of cancer patients, and may also be detected in lymph nodes affected by cancer cells or minimal residual disease cells either locally deposited (e.g. bone marrow, liver, kidney) or freely floating throughout the patients body. The sample to be analyzed can be tissue material from a neoplastic lesion taken by aspiration or punctuation, excision or by any other surgical method leading to biopsy or resected cellular material. The sample might comprise cells obtained from the patient. The cells may be found in a cell “smear” collected, for example, by a fine needle biopsy or from provoked or spontaneous nipple discharge. Another example of a sample is a body fluid. Such body fluids include, for example, blood fluids, lymph, ascitic fluids, gynecological fluids, or urine but not limited to these fluids.

In the method of the present invention the determination of gene expression (or the determination of the pattern of expression levels) is not limited to any specific method or to the detection of mRNA.

The presence and/or level of expression of one or more marker genes in a sample can be assessed, for example, by measuring and/or quantifying:

(a) a protein encoded by a marker gene in Table 1 or a polypeptide resulting from the processing or degradation of the protein (e.g. using a reagent, such as an antibody, an antibody derivative, or an antibody fragment, which binds specifically with the protein or polypeptide);
(b) a metabolite which is produced directly (i.e., catalyzed) or indirectly by a protein encoded by a marker gene in Table 1 or by a polypeptide encoded thereby; or
(c) an RNA transcript (e.g., mRNA, hnRNA) encoded by a marker gene in Table 1, or a fragment of the RNA transcript (e.g. by contacting a mixture of RNA transcripts obtained from the sample or cDNA prepared from the transcripts with a nucleic acid probe comprising a sequence of one or more of the marker genes listed within Table 1 fixed thereto at selected positions). The mRNA expression of these genes can be detected e.g. with DNA-microarray as provided by Affymetrix Inc. (U.S. Pat. No. 5,556,752) or other manufacturers. For example, the expression of these genes can be detected with bead based direct fluorescent readout techniques such as provided by Luminex Inc. (WO 97/14028).

In a preferred embodiment of the method of the present invention, in step (b) the pattern of expression levels of at least three marker genes of Table 1 is determined.

In a more preferred embodiment of the method of the present invention, in step (b) the pattern of expression levels of at least six marker genes of Table 1 is determined.

In an even more preferred embodiment of the method of the present invention comprises the following steps:

(a) obtaining a biological sample from a patient;
(b) determining at least the pattern of expression levels of VEGFC, ERBB3 and/or Her2/neu;
(c) comparing the pattern of expression levels determined in (b) with one or several reference pattern(s) of expression levels;
wherein (i) upregulated expression of VEGFC and/or (ii) downregulated expression of ERBB3 and/or Her2/neu is indicative of a poor prognosis in regard to therapeutic success for said given mode of treatment in said subject.

In a preferred embodiment of the method of the invention said treatment (a) acts on recruitment of lymphatic vessels, cell proliferation, cell survival and/or cell motility, and/or (b) comprises administration of a chemotherapeutic agent.

Particularly preferred are modes of treatment comprising chemotherapy, administration of small molecule inhibitors, antibody based regimens, anti-proliferation regimens, pro-apoptotic regimens, pro-differentiation regimens, radiation and/or surgical therapy. Thus, the methods of the invention may be used to evaluate a patient before, during and after therapy, for example, to evaluate the reduction in tumor burden.

In a further aspect, the present invention provides a method for selecting a therapy regimen (e.g. the kind of chemotherapeutic argents) for inhibiting cancer in a patient comprising the steps of:

(a) obtaining a biological sample from said patient;
(b) predicting from said sample, by a method of the present invention as discussed above therapeutic success for a plurality of individual modes of treatment; and
(c) selecting a mode of treatment which is predicted to be successful in step (b).

In a preferred embodiment, said method comprises—in addition to step (a)—the following steps:

b) separately maintaining aliquots of the sample in the presence of one or more test compositions;
c) comparing expression of a single or plurality of marker genes, selected from the marker genes listed in Table 1 in each of the aliquots; and
d) selecting a test composition which induces a lower level of expression of genes from Table 1 and/or a higher level of expression of genes from Table 1 in the aliquot containing that test composition, relative to the level of expression of each marker gene in the aliquots containing the other test compositions.

The invention also provides a kit useful for carrying out a method of the invention, comprising at least (a₁) three primer pairs and/or (a₂) three probes each having a sequence sufficiently complementary to the genes encoding VEGFC, ERBB3 and/or Her2/neu and/or (b) at least three antibodies directed against VEGFC, ERBB3 and Her2/neu.

Finally, the present invention relates to the use of (a) an anti-VEGFC antibody, (b) an antisense nucleic acid or a ribozyme inhibiting the expression of the VEGFC encoding gene or (c) an inactive version of VEGFC for the preparation of a pharmaceutical composition for the treatment of a cancer associated with the recruitment of lymphatic vessels by the expression of VEGFC, preferably HNSCC, breast or colon cancer.

### BRIEF DESCRIPTION OF THE DRAWINGS

FIG. 1: Relative expression of candidate genes as determined by qRT-PCR profiling in head and neck cancer and grouping of samples on the basis of overall survival after primary surgery

FIG. 2: Relative expression of 3 candidate genes (VEGFC, ERBB3 and Her-2/neu) in a Finding Cohort (most extreme cases)

as determined by qRT-PCR profiling in head and neck cancer and grouping of samples on the basis of overall survival after primary surgery

FIG. 3: Relative expression of candidate genes (ERBB Family, Keratins 5 and 14, VEGF alpha isoforms and VEGFC) in the total cohort for verification of trends seen in the Finding Cohort

as determined by qRT-PCR profiling in head and neck cancer and grouping of samples on the basis of overall survival after primary surgery

FIG. 4: Principal component analysis based on relative expression of 3 candidate genes (VEGFC, ERBB3 and Her-2/neu)

as determined by qRT-PCR profiling in head and neck cancer and grouping of samples on the basis of overall survival after primary surgery

FIG. 5: Relative expression of candidate genes as determined by Affymetrix gene expression profiling in head and neck cancer and grouping of samples on the basis of overall survival after primary surgery. Proof of the discriminative power of VEGFC, VEGFB, ERBB2 and ERBB3. Affymetrix platform restrictions clearly visible according to lack of performance of several probe sets.

FIG. 6: Illustration of the process for model generation and cross-validation

FIG. 7: Classification based on K-nearest neighbour analysis based on the relative expression of candidate genes as determined by qRT-PCR profiling in head and neck cancer and grouping of samples on the basis of overall survival after primary surgery.

FIG. 8: Kaplan-Meier-Analysis of overall survival (OAS)

based on relative gene expression of VEGFC as determined by qRT-PCR. Expression level cut-off criteria are set at 600 relative gene copies.

FIG. 9: Kaplan-Meier-Analysis of overall survival (OAS)

based on relative gene expression of Her-2/neu as determined by qRT-PCR. Expression level cut-off criteria are set at 400 relative gene copies.

FIG. 10: Kaplan-Meier-Analysis of overall survival (OAS) based on relative gene expression of ERBB3 as determined by qRT-PCR. Expression level cut-off criteria are set at 900 relative gene copies.

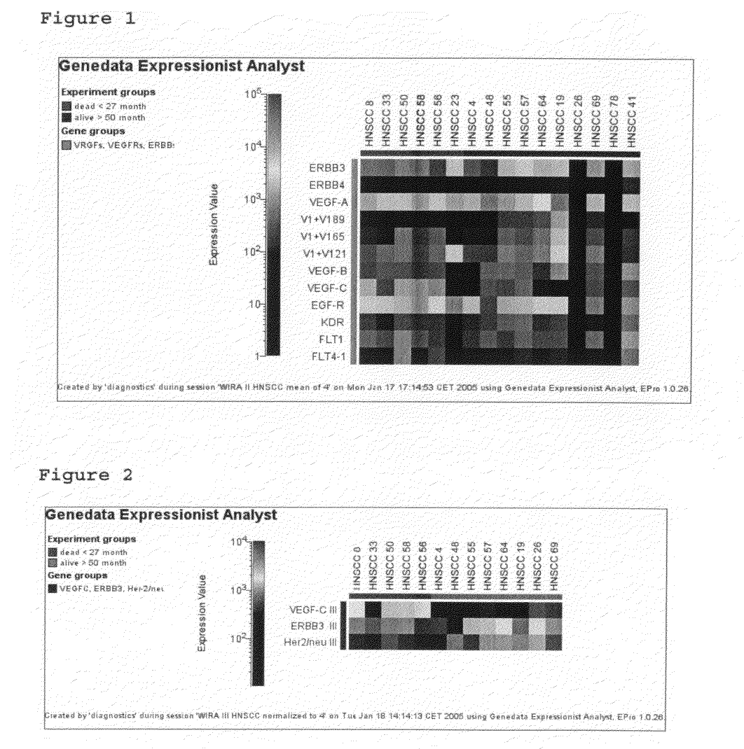

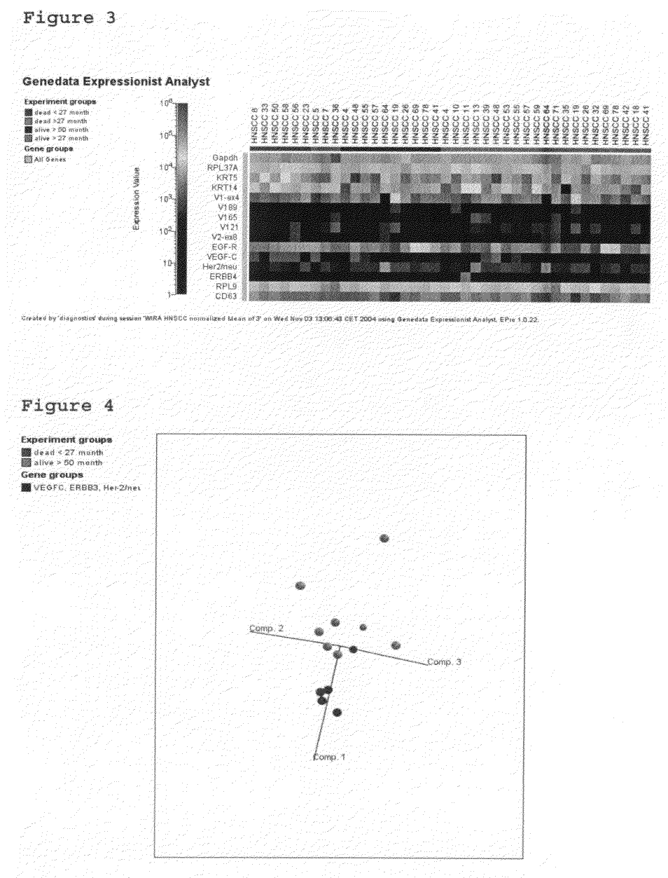

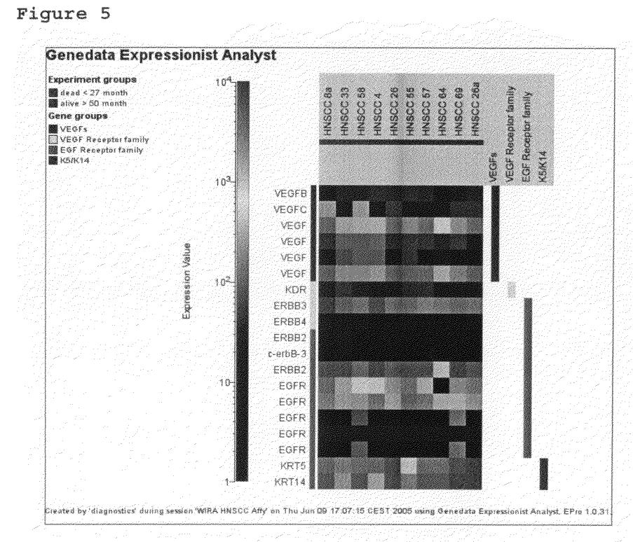

### EXAMPLE 1

### General Methods

### (A) Experimental Procedures and Settings

Modes of treatment comprise chemotherapy (5-FU based, anthracycline based), small molecule inhibitors (Iressa, Sorafenib, SU 11248), antibody based regimens (Trastuzumab, avastin).

Cytotoxic and cytostatic agents are common therapeutics for advanced head and neck, colon and breast cancer. These compounds have been established as important chemotherapeutic agents in the armamentarium of drugs to treat cancer in the 1970s and are still in use. Expression profiles of 60 fresh frozen surgical resectates of advanced head and neck cancer have been obtained by the use of RT-PCR strategies and oligonucleotide microarrays (Affymetrix). 49 tumors were used for marker identification approaches. In addition 24 non-advanced head and neck cancer resectates were available for analysis.

Analyzing the data for 49 advanced tumors by statistical methods as described in Examples 2 and 3, 12 significantly differentially expressed genes (listed in Table 1) were identified.

### (B) Biological Relevance of Genes of Table 1

Some of the genes listed in Table 1 represent biological, cellular processes and are characterized by similar regulation mechanisms. Some characteristic genes from Table 1 are described here in greater detail:

### VEGFC

The process of angiogenesis is regulated by vascular endothelial growth factor (VEGF) and its 2 known receptor tyrosine kinases FLT1 and KDR/FLK1. The receptor tyrosine kinase FLT4 is expressed mainly in lymphatic endothelia but does not bind VEGF. Affinity chromatography was used to isolate the ligand of FLT4. It was found to be a polypeptide of 23 kD and its N-terminal protein sequence was determined. Degenerate oligonucleotides based on this N-terminal sequence were used to clone the corresponding cDNA from a human PC-3 cell cDNA library. The resulting clone was named VEGFC. VEGFC was cloned from a human glioma G61 cell cDNA library using a probe based on a sequence from the EST library. Sequence analysis showed that the full-length clones contained an open reading frame of 350 amino acids with a VEGF-homologous region that is 30% identical to VEGF and 27% identical to VEGFB/VRF. The N-terminus contains a putative secretory signal sequence. It has been noted that the C-terminus of VEGFC has cysteine-rich repeat units characteristic of the Balbiani ring 3 protein (BR3P) of the midge Chironomus tentans. Transfection assays suggested that VEGFC forms disulfide-linked dimers and can activate both the FLT4 and KDR/FLK1 receptor tyrosine kinases. Competitive binding assays of purified components showed that VEGFC and FLT4 bind with high affinity, suggesting that VEGFC is a biologically relevant ligand of FLT4. It was also demonstrated that conditioned medium from cells expressing VEGFC could stimulate the growth of endothelial cells in a collagen gel matrix.

The differential expression of VEGFC might explain the different propensity to lymph node metastasis in thyroid cancers. Using real-time quantitative PCR, 111 normal and neoplastic thyroid tissues were analyzed. Papillary thyroid cancers had a higher VEGFC expression than other thyroid malignancies (P less than 0.0005 ANOVA). Paired comparison of VEGFC expression between thyroid cancers and normal thyroid tissues from the same patients showed a significant increase of VEGFC expression in papillary thyroid cancer and a significant decrease of VEGFC expression in medullary thyroid cancer. In contrast, there was no significant difference of VEGFC expression between cancer and normal tissues in other types of thyroid cancer.

### Her2/neu

The oncogene originally called NEU was derived from rat neuro/glioblastoma cell lines. It encodes a tumor antigen, p185, which is serologically related to EGFR, the epidermal growth factor receptor. EGFR maps to chromosome 7. In 1985 it was found, that the human homologue, which was designated NGL (to avoid confusion with neuraminidase, which is also symbolized NEU), maps to 17q12-q22 by in situ hybridization and to 17q21-qter in somatic cell hybrids. Thus, the SRO is 17q21-q22. Moreover, in 1985 a potential cell surface receptor of the tyrosine kinase gene family was identified and characterized by cloning the gene. Its primary sequence is very similar to that of the human epidermal growth factor receptor. Because of the seemingly close relationship to the human EGF receptor, the authors called the gene HER2. By Southern blot analysis of somatic cell hybrid DNA and by in situ hybridization, the gene was assigned to 17q21-q22. This chromosomal location of the gene is coincident with the NEU oncogene, which suggests that the 2 genes may in fact be the same; indeed, sequencing indicates that they are identical. In 1988 a correlation between over expression of NEU protein and the large-cell, comedo growth type of ductal carcinoma was found. The authors found no correlation, however, with lymph-node status or tumor recurrence. The role of HER2/NEU in breast and ovarian cancer was described in 1989, which together account for one-third of all cancers in women and approximately one-quarter of cancer-related deaths in females.

An ERBB-related gene that is distinct from the ERBB gene, called ERBB1 was found in 1985. ERBB2 was not amplified in vulva carcinoma cells with EGFR amplification and did not react with EGF receptor mRNA. About 30-fold amplification of ERBB2 was observed in a human adenocarcinoma of the salivary gland. By chromosome sorting combined with velocity sedimentation and Southern hybridization, the ERBB2 gene was assigned to chromosome 17. By hybridization to sorted chromosomes and to metaphase spreads with a genomic probe, they mapped the ERBB2 locus to 17q21. This is the chromosome 17 breakpoint in acute promyelocytic leukemia (APL). Furthermore, amplification and elevated expression of the ERBB2 gene was observed in a gastric cancer cell line. Antibodies against a synthetic peptide corresponding to 14 amino acid residues at the COOH-terminus of a protein deduced from the ERBB2 nucleotide sequence were raised in 1986. With these antibodies, the ERBB2 gene product from adenocarcinoma cells was precipitated and demonstrated to be a 185-kD glycoprotein with tyrosine kinase activity. A cDNA probe for ERBB2 and by in situ hybridization to APL cells with a 15;17 chromosome translocation located the gene to the proximal side of the breakpoint. The authors suggested that both the gene and the breakpoint are located in band 17q21.1 and, further, that the ERBB2 gene is involved in the development of leukemia. In 1987 experiments indicated that NEU and HER2 are both the same as ERBB2. The authors demonstrated that over expression alone can convert the gene for a normal growth factor receptor, namely, ERBB2, into an oncogene. The ERBB2 was mapped to 17q11-q21 by in situ hybridization. By in situ hybridization to chromosomes derived from fibroblasts carrying a constitutional translocation between 15 and 17, they showed that the ERBB2 gene was relocated to the derivative chromosome 15; the gene can thus be localized to 17q12-q21.32. By family linkage studies using multiple DNA markers in the 17q12-q21 region the ERBB2 gene was placed on the genetic map of the region.

Interleukin-6 is a cytokine that was initially recognized as a regulator of immune and inflammatory responses, but also regulates the growth of many tumor cells, including prostate cancer. Over expression of ERBB2 and ERBB3 has been implicated in the neoplastic transformation of prostate cancer. Treatment of a prostate cancer cell line with IL6 induced tyrosine phosphorylation of ERBB2 and ERBB3, but not ERBB1/EGFR. The ERBB2 forms a complex with the gp130 subunit of the IL6 receptor in an IL6-dependent manner. This association was important because the inhibition of ERBB2 activity resulted in abrogation of the IL6-induced MAPK activation. Thus, ERBB2 is a critical component of IL6 signaling through the MAP kinase pathway. These findings showed how a cytokine receptor can diversify its signaling pathways by engaging with a growth factor receptor kinase.

Over expression of ERBB2 confers Taxol resistance in breast cancer. Over expression of ERBB2 inhibits Taxol-induced apoptosis. Taxol activates the CDC2 kinase in MDA-MB-435 breast cancer cells, leading to cell cycle arrest at the G2/M phase and, subsequently, apoptosis. A chemical inhibitor of CDC2 and a dominant-negative mutant of CDC2 blocked Taxol-induced apoptosis in these cells. Over expression of ERBB2 in MDA-MB-435 cells by transfection transcriptionally upregulates CDKN1A which associates with CDC2, inhibits Taxol-mediated CDC2 activation, delays cell entrance to G2/M phase, and thereby inhibits Taxol-induced apoptosis. In CDKN1A antisense-transfected MDA-MB-435 cells or in p21−/− MEF cells, ERBB2 was unable to inhibit Taxol-induced apoptosis. Therefore, CDKN1A participates in the regulation of a G2/M checkpoint that contributes to resistance to Taxol-induced apoptosis in ERBB2-overexpressing breast cancer cells.

A secreted protein of approximately 68 kD was described, designated herstatin, as the product of an alternative ERBB2 transcript that retains intron 8. This alternative transcript specifies 340 residues identical to subdomains I and II from the extracellular domain of p185ERBB2, followed by a unique C-terminal sequence of 79 amino acids encoded by intron 8. The recombinant product of the alternative transcript specifically bound to ERBB2-transfected cells and was chemically crosslinked to p185ERBB2, whereas the intron-encoded sequence alone also bound with high affinity to transfected cells and associated with p185 solubilized from cell extracts. The herstatin mRNA was expressed in normal human fetal kidney and liver, but was at reduced levels relative to p185ERBB2 mRNA in carcinoma cells that contained an amplified ERBB2 gene. Herstatin appears to be an inhibitor of p185ERBB2, because it disrupts dimers, reduces tyrosine phosphorylation of p185, and inhibits the anchorage-independent growth of transformed cells that overexpress ERBB2. The HER2 gene is amplified and HER2 is overexpressed in 25 to 30% of breast cancers, increasing the aggressiveness of the tumor. Finally, it was found that a recombinant monoclonal antibody against HER2 increased the clinical benefit of first-line chemotherapy in metastatic breast cancer that overexpresses HER2.

### ERBB3

In 1989 a DNA fragment related to but distinct from epidermal growth factor receptor EGFR and ERBB2 was detected. cDNA cloning showed a predicted 148 kD transmembrane polypeptide with structural features identifying it as a member of the ERBB gene family, prompting the designation ERBB3. Markedly elevated ERBB3 mRNA levels were demonstrated in certain human mammary tumor cell lines, suggesting that it may play a role in some human malignancies just as EGFR (also called ERBB1) does. Epidermal growth factor, transforming growth factor alpha and amphiregulin are structurally and functionally related growth regulatory proteins. They are all polypeptides that bind to the 170 kD cell-surface EGF receptor, activating its intrinsic kinase activity. These 3 proteins differentially interact with a homolog of EGFR. They failed to show any interaction between these 3 secreted growth factors and ERBB2, a known EGFR-related protein. Searching for other members of this family of receptor tyrosine kinases, however, they cloned and studied the expression of ERBB3, which they referred to as HER3. The cDNA was isolated from a human carcinoma cell line, and its 6-kb transcript was identified in various human tissues. ERBB3 is a receptor for heregulin and is capable of mediating HGL-stimulated tyrosine phosphorylation of itself. The 2.6-angstrom crystal structure of the entire extracellular region of human HER3 has been determined. The structure consists of 4 domains with structural homology to domains found in the type I insulin-like growth factor receptor. The HER3 structure revealed a contact between domains II and IV that constrains the relative orientations of ligand-binding domains and provides a structural basis for understanding both multiple-affinity forms of EGFRs and conformational changes induced in the receptor by ligand binding during signaling. By in situ hybridization ERBB3 gene has been mapped to chromosome 12q13.

### ERBB4

The HER4/ERBB4 gene is a member of the type I receptor tyrosine kinase subfamily that includes EGFR, ERBB2, and ERBB3. It encodes a receptor for NDF/heregulin (NRG1). Using in situ hybridization and immunohistochemical analysis, it was shown that Erbb4 was extensively expressed in adult and fetal mouse tissues. Expression was strong in the lining epithelia of the gastrointestinal, urinary, reproductive, and respiratory tracts, as well as in skin, skeletal muscle, circulatory, endocrine, and nervous systems. The developing brain and heart expressed high levels of Erbb4. Neuregulins and their receptors, the ERBB protein tyrosine kinases, are essential for neuronal development. ERBB4 is enriched in the postsynaptic density and associates with PSD95. Heterologous expression of PSD95 enhanced NRG activation of ERBB4 and MAP kinase. Conversely, inhibiting expression of PSD95 in neurons attenuated NRG-mediated activation of MAP kinase. PSD95 formed a ternary complex with 2 molecules of ERBB4, suggesting that PSD95 facilitates ERBB4 dimerization. Finally, NRG suppressed induction of long-term potentiation in the hippocampal CA1 region without affecting basal synaptic transmission. Thus, NRG signaling may be synaptic and regulated by PSD95. The role of NRG signaling in the adult central nervous system may be the modulation of synaptic plasticity. ERBB4 and PSD95 co-immunoprecipitated from rat forebrain lysates and this direct interaction was mediated through the C-terminal end of ERBB4. Immunofluorescent studies of cultured rat hippocampal cells showed that ERBB4 co-localized with PSD95 and NMDA receptors at interneuronal postsynaptic sites. The findings suggested that certain ERBB receptors interact with other receptors and may be important in activity-dependent synaptic plasticity. ERBB4 is a transmembrane receptor tyrosine kinase that regulates cell proliferation and differentiation. After binding its ligand, heregulin, or activation of protein kinase C by TPA, the ERBB4 ectodomain is cleaved by a metalloprotease. Subsequent cleavage by gamma-secretase releases the ERBB4 intracellular domain from the membrane and facilitates its translocation to the nucleus. Gamma-secretase cleavage was prevented by chemical inhibitors or a dominant-negative presenilin. Inhibition of gamma-secretase also prevented growth inhibition by heregulin. Gamma-secretase cleavage of ERBB4 may represent another mechanism for receptor tyrosine kinase-mediated signaling. Using human cDNA probes in fluorescence in situ hybridization the ERBB4 gene has been mapped to chromosome 2q33.3-q34. The finding established that the ERBB4 gene, like the related EGFR, ERBB2, and ERBB3 genes, is located in close proximity to homeobox and collagen gene loci. ErbB4−/− mouse embryos develop trigeminal ganglion and geniculate/cochleovestibular ganglia that are displaced toward each other and show axonal misprojections. These morphologic changes correlate with aberrant migration of a subpopulation of hindbrain-derived cranial neural crest cells. The aberrant migration is also accompanied by an apparent downregulation of HoxB2 gene expression. Through transplantation experiments, it was determined that neural crest cells deviated from their normal pathway only when transplanted into mutant embryos, suggesting that ErbB4 signaling within the host environment provides patterning information essential for the proper migration of neural crest cells. Transgenic mice were generated that expressed a dominant-negative ErbB4 receptor specifically in non-myelinating Schwann cells. The mutant mice developed a progressive peripheral neuropathy characterized by extensive Schwann cell proliferation and death, loss of un-myelinated axons, and marked hot and cold pain insensitivity. At later stages, the mutant mice showed a loss of C-fiber dorsal root ganglion neurons. The findings indicated that the NRG1-ErbB4 signaling system contributes to reciprocal interactions between un-myelinated sensory axons and non-myelinating Schwann cells that appear to be critical for Schwann cell and C-fiber sensory neuron survival. ERBB4 was expressed at high levels in neural precursor cells in the rat subventricular zone (SVZ) and rostral migratory system (RMS) that are destined to become olfactory interneurons. ERBB4 was also detected in a subset of glial cells. Mice with targeted deletion of the ErbB4 gene in the CNS showed cellular disorganization of the SVZ and RMS as well as altered distribution and differentiation of olfactory interneurons. In vivo, cells explanted from mutant mice failed to form migratory neuronal chains and showed impaired orientation compared to wildtype cells. It has been concluded that ERBB4 plays a role in RMS neuroblast tangential migration and olfactory interneuronal placement.

Mice lacking neural Erbb4 expression had reduced numbers of GABA-positive neurons in the postnatal cortex and hippocampus. Nrg1 is a neural guidance molecule for GABAergic interneurons from the medial ganglionic eminence. Thus, the loss of GABAergic neurons in Erbb4 mutant mice was attributed to abnormal migration of these interneurons to the neocortex.

### (C) Identification of Differential Expression

Transcripts within the collected RNA samples which represent RNA produced by differentially expressed genes may be identified by utilizing a variety of methods which are well known to those of skill in the art. For example, differential screening (Tedder et al., PNAS USA 85 (1988), 208-212), subtractive hybridization (Hedrick et al., Nature 308 (1984), 149-53) and, preferably, differential display (U.S. Pat. No. 5,262,311) may be utilized to identify polynucleotide sequences derived from genes that are differentially expressed.

Differential screening involves the duplicate screening of a cDNA library in which one copy of the library is screened with a total cell cDNA probe corresponding to the mRNA population of one cell type while a duplicate copy of the cDNA library is screened with a total cDNA probe corresponding to the mRNA population of a second cell type. For example, one cDNA probe may correspond to a total cell cDNA probe of a cell type derived from a control subject, while the second cDNA probe may correspond to a total cell cDNA probe of the same cell type derived from an experimental subject. Those clones which hybridize to one probe but not to the other potentially represent clones derived from genes differentially expressed in the cell type of interest in control versus experimental subjects.

Subtractive hybridization techniques generally involve the isolation of mRNA taken from two different sources, e.g., control and experimental tissue, the hybridization of the mRNA or single-stranded cDNA reverse-transcribed from the isolated mRNA, and the removal of all hybridized, and therefore double-stranded, sequences. The remaining non-hybridized, single-stranded cDNA, potentially represent clones derived from genes that are differentially expressed in the two mRNA sources. Such single-stranded cDNA is then used as the starting material for the construction of a library comprising clones derived from differentially expressed genes.

The differential display technique describes a procedure, utilizing the well known polymerase chain reaction (U.S. Pat. No. 4,683,202) which allows for the identification of sequences derived from genes which are differentially expressed. First, isolated RNA is reverse-transcribed into single-stranded cDNA, utilizing standard techniques which are well known to those of skill in the art. Primers for the reverse transcriptase reaction may include, but are not limited to, oligo dT-containing primers, preferably of the reverse primer type of oligonucleotides described below. Next, this technique uses pairs of PCR primers, as described below, which allows for the amplification of clones representing a random subset of the RNA transcripts present within any given cell. Utilizing different pairs of primers allows each of the mRNA transcripts present in a cell to be amplified. Among such amplified transcripts those may be identified which have been produced from differentially expressed genes.

The reverse oligonucleotide primer of the primer pairs may contain an oligo dT stretch of nucleotides, preferably eleven nucleotides long, at its 5′ end, which hybridizes to the poly(A) tail of mRNA or to the complement of a cDNA reverse transcribed from an mRNA poly(A) tail. Second, in order to increase the specificity of the reverse primer, the primer may contain one or more, preferably two, additional nucleotides at its 3′ end. Because, statistically, only a subset of the mRNA derived sequences present in the sample of interest will hybridize to such primers, the additional nucleotides allow the primers to amplify only a subset of the mRNA derived sequences present in the sample of interest. This is preferred in that it allows more accurate and complete visualization and characterization of each of the bands representing amplified sequences.

The forward primer may contain a nucleotide sequence expected, statistically, to have the ability to hybridize to cDNA sequences derived from the tissues of interest. The nucleotide sequence may be an arbitrary one, and the length of the forward oligonucleotide primer may range from about 9 to about 13 nucleotides, with about 10 nucleotides being preferred. Arbitrary primer sequences cause the lengths of the amplified partial cDNAs produced to be variable, thus allowing different clones to be separated by using standard denaturing sequencing gel electrophoresis. PCR reaction conditions should be chosen which optimize amplified product yield and specificity, and, additionally, produce amplified products of lengths which may be resolved utilizing standard gel electrophoresis techniques. Such reaction conditions are well known to those of skill in the art, and important reaction parameters include, for example, length and nucleotide sequence of oligonucleotide primers as discussed above, and annealing and elongation step temperatures and reaction times. The pattern of clones resulting from the reverse transcription and amplification of the mRNA of two different cell types is displayed via sequencing gel electrophoresis and compared. Differences in the two banding patterns indicate potentially differentially expressed genes. When screening for full-length cDNAs, it is preferable to use libraries that have been size-selected to include larger cDNAs. Randomly-primed libraries are preferable, in that they will contain more sequences which contain the 5′ regions of genes. Use of a randomly-primed library may be especially preferable for situations in which an oligo d(T) library does not yield a full-length cDNA. Genomic libraries can be useful for extension of sequence into 5′ nontranscribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) which are laser activated, and detection of the emitted wavelengths by a charge coupled device camera. output/light intensity can be converted to electrical signal using appropriate software (e.g. GENOTYPER and Sequence NAVIGATOR, Perkin Elmer; ABI), and the entire process from loading of samples to computer analysis and electronic data display can be computer controlled. Capillary electrophoresis is especially preferable for the sequencing of small pieces of DNA which might be present in limited amounts in a particular sample.

Once potentially differentially expressed gene sequences have been identified via bulk techniques such as, for example, those described above, the differential expression of such putatively differentially expressed genes should be corroborated. Corroboration may be accomplished via, for example, such well known techniques as Northern analysis and/or RT-PCR. Upon corroboration, the differentially expressed genes may be further characterized, and may be identified as target and/or marker genes, as discussed, below.

Also, amplified sequences of differentially expressed genes obtained through, for example, differential display may be used to isolate full length clones of the corresponding gene. The full length coding portion of the gene may readily be isolated, without undue experimentation, by molecular biological techniques well known in the art. For example, the isolated differentially expressed amplified fragment may be labeled and used to screen a cDNA library. Alternatively, the labeled fragment may be used to screen a genomic library.

An analysis of the tissue distribution of the mRNA produced by the identified genes may be conducted, utilizing standard techniques well known to those of skill in the art. Such techniques may include, for example, Northern analyses and RT-PCR. Such analyses provide information as to whether the identified genes are expressed in tissues expected to contribute to cancer. Such analyses may also provide quantitative information regarding steady state mRNA regulation, yielding data concerning which of the identified genes exhibits a high level of regulation in, preferably, tissues which may be expected to contribute to cancer.

Such analyses may also be performed on an isolated cell population of a particular cell type derived from a given tissue. Additionally, standard in situ hybridization techniques may be utilized to provide information regarding which cells within a given tissue express the identified gene. Such analyses may provide information regarding the biological function of an identified gene relative to cancer in instances wherein only a subset of the cells within the tissue is thought to be relevant to cancer.

### (D) Identification of Polynucleotide Variants and Homologues or Splice Variants

Variants and homologues of the “CANCER GENE” polynucleotides described above also are “CANCER GENE” polynucleotides. Typically, homologous “CANCER GENE” polynucleotide sequences can be identified by hybridization of candidate polynucleotides to known “CANCER GENE” polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50 EC once, 30 minutes; then 2×SSC, room temperature twice, 10 minutes each homologous sequences can be identified which contain at most about 25-30% base pair mismatches. More preferably, homologous polynucleotide strands contain 15-25% base pair mismatches, even more preferably 5-15% base pair mismatches.

Species homologues of the “CANCER GENE” polynucleotides disclosed herein can also be identified by making suitable probes or primers and screening cDNA expression libraries from other species, such as mice, monkeys, or yeast. Human variants of “CANCER GENE” polynucleotides can be identified, for example, by screening human cDNA expression libraries. It is well known that the Tₘ of a double-stranded DNA decreases by 1-1.5° C. with every 1% decrease in homology (Bonner et al., J. Mol. Biol. 81 (1973), 123). Variants of human “CANCER GENE” polynucleotides or “CANCER GENE” polynucleotides of other species can therefore be identified by hybridizing a putative homologous “CANCER GENE” polynucleotide with a polynucleotide having a nucleotide sequence of one of the genes of the Table 1 or the complement thereof to form a test hybrid. The melting temperature of the test hybrid is compared with the melting temperature of a hybrid comprising polynucleotides having perfectly complementary nucleotide sequences, and the number or percent of base pair mismatches within the test hybrid is calculated.

Nucleotide sequences which hybridize to “CANCER GENE” polynucleotides or their complements following stringent hybridization and/or wash conditions also are “CANCER GENE” polynucleotides. Stringent wash conditions are well known and understood in the art and are disclosed, for example, in Sambrook et al., Molecular Cloning: A Laboratory Manual, 2d ed. (1989); Ausubel et al., Current Protocols In Molecular Biology, John Wiley & Sons, New York, N.Y. (1989). Typically, for stringent hybridization conditions a combination of temperature and salt concentration should be chosen that is approximately 12 to 20° C. below the calculated Tₘ of the hybrid under study. The Tₘ of a hybrid between a “CANCER GENE” polynucleotide having a nucleotide sequence of one of the sequences of Table 1 or the complement thereof and a polynucleotide sequence which is at least about 50, preferably about 75, 90, 96, or 98% identical to one of those nucleotide sequences can be calculated, for example, using the equation below [Bolton and McCarthy, 1962, (11):

Tₘ=81.5° C.−16.6(log₁₀[Na⁺])+0.41(% G+C)−0.63(% formamide)−600/l,

where l=the length of the hybrid in base pairs. Stringent wash conditions include, for example, 4×SSC at 65° C., or 50% formamide, 4×SSC at 28° C., or 0.5×SSC, 0.1% SDS at 65° C. Highly stringent wash conditions include, for example, 0.2×SSC at 65° C.

### (E) Detecting Expression and Gene Product

Although the presence of marker gene expression suggests that the “CANCER GENE” polynucleotide is also present, its presence and expression may need to be confirmed. For example, if a sequence encoding a “CANCER GENE” polypeptide is inserted within a marker gene sequence, transformed cells containing sequences which encode a “CANCER GENE” polypeptide can be identified by the absence of marker gene function. Alternatively, a marker gene can be placed in tandem with a sequence encoding a “CANCER GENE” polypeptide under the control of a single promoter. Expression of the marker gene in response to induction or selection usually indicates expression of the “CANCER GENE” polynucleotide.

Alternatively, host cells which contain a “CANCER GENE” polynucleotide and which express a “CANCER GENE” polypeptide can be identified by a variety of procedures known to those of skill in the art. These procedures include DNA-DNA or DNA-RNA hybridization and protein bioassay or immunoassay techniques which include membrane, solution, or chip-based technologies for the detection and/or quantification of polynucleotides or proteins. For example, the presence of a polynucleotide sequence encoding a “CANCER GENE” polypeptide can be detected by DNA-DNA or DNA-RNA hybridization or amplification using probes or fragments or fragments of polynucleotides encoding a “CANCER GENE” polypeptide. Nucleic acid amplification-based assays involve the use of oligonucleotides selected from sequences encoding a “CANCER GENE” polypeptide to detect transformants which contain a “CANCER GENE” polynucleotide.

A variety of protocols for detecting and measuring the expression of a “CANCER GENE” polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a “CANCER GENE” polypeptide can be used, or a competitive binding assay can be employed. These and other assays are described in Hampton et al., SEROLOCICAL METHODS: A LABORATORY MANUAL, APS Press, St. Paul, Minn., 1990.

A wide variety of labels and conjugation techniques are known by those skilled in the art and can be used in various nucleic acid and amino acid assays. Means for producing labeled hybridization or PCR probes for detecting sequences related to polynucleotides encoding “CANCER GENE” polypeptides include oligo labeling, nick translation, end-labeling, or PCR amplification using a labeled nucleotide. Alternatively, sequences encoding a “CANCER GENE” polypeptide can be cloned into a vector for the production of an mRNA probe. Such vectors are known in the art, are commercially available, and can be used to synthesize RNA probes in vitro by the addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

### (F) Predictive, Diagnostic and Prognostic Assays

Biological samples can be screened for the presence and/or absence of the biomarkers identified herein. Such samples are for example needle biopsy cores, surgical resection samples, or body fluids like serum, thin needle nipple aspirates and urine. For example, these methods include obtaining a biopsy, which is optionally fractionated by cryostat sectioning to enrich disease cells to about 80% of the total cell population. In certain embodiments, polynucleotides extracted from these samples may be amplified using techniques well known in the art. The expression levels of selected markers detected would be compared with statistically valid groups of diseased and healthy samples.

Abnormal mRNA and/or protein level of the disclosed markers can be determined by various well-known methods, such as Northern blot analysis, reverse transcription polymerase chain reaction (RT-PCR), in situ hybridization, immunoprecipitation, Western blot hybridization, or immuno-histochemistry. According to the method, cells are obtained from a test subject and the levels of the disclosed biomarkers, proteins or mRNA are determined and compared to the level of these markers in a healthy subject. An abnormal level of the biomarker polypeptide or mRNA levels is likely to be indicative of malignant neoplasia such as head and neck, colon or breast cancer. Further methods are Southern blot analysis, dot blot analysis, Fluorescence or colorimetric in situ hybridization, comparative genomic hybridization or quantitative PCR. In general these assays comprise the usage of probes from representative genomic regions. The probes contain at least parts of said genomic regions or sequences complementary or analogous to said regions, in particular intra- or intergenic regions of said genes or genomic regions. The probes can consist of nucleotide sequences or sequences of analogous functions (e.g. PNAs, morpholino-oligomers) being able to bind to target regions by hybridization. In general genomic regions being altered in said patient samples are compared with unaffected control samples (normal tissue from the same or different patients, surrounding unaffected tissue, peripheral blood) or with genomic regions of the same sample that don't have said alterations and can therefore serve as internal controls. In a preferred embodiment regions located on the same chromosome are used. Alternatively, gonosomal regions and/or regions with defined varying amounts in the sample are used. In one favored embodiment the DNA content, structure, composition or modification that lie within distinct genomic regions are compared. Especially favored are methods that detect the DNA content of said samples, where the amounts of target regions are altered by amplification and/or deletions. In another embodiment the target regions are analyzed for the presence of polymorphisms (e.g. single nucleotide polymorphisms or mutations) that affect or predispose the cells in said samples with regard to clinical aspects being of diagnostic, prognostic or therapeutic value. Preferably, the identification of sequence variations is used to define haplotypes that result in a characteristic behavior of said samples with said clinical aspects.

### (G) DNA Array Technology

Polynucleotide probes can be immobilized on a DNA chip in an organized array. Oligonucleotides can be bound to a solid support by a variety of processes, including lithography. For example, a chip can hold up to 410.000 oligonucleotides (GeneChip, Affymetrix). The present invention provides significant advantages over the available tests for malignant neoplasia, such as head and neck, colon or breast cancer, because it increases the reliability of the test by providing an array of polynucleotide markers an a single chip.

The method includes obtaining a biologocal sample which can be a biopsy of an affected person, which is optionally fractionated by cryostat sectioning to enrich diseased cells to about 80% of the total cell population and the use of body fluids such as serum, urine, or cell containing fluids(e.g. derived from fine needle aspirates). The DNA or RNA is then extracted, amplified, and analyzed with a DNA chip to determine the presence of absence of the marker polynucleotide sequences. In one embodiment, the polynucleotide probes are spotted onto a substrate in a two-dimensional matrix or array. Samples of polynucleotides can be labeled and then hybridized to the probes. Double-stranded polynucleotides, comprising the labeled sample polynucleotides bound to probe polynucleotides, can be detected once the unbound portion of the sample is washed away.

The probe polynucleotides can be spotted on substrates including glass, nitrocellulose, etc. The probes can be bound to the substrate by either covalent bonds or by non-specific interactions, such as hydrophobic interactions. The sample polynucleotides can be labeled using radioactive labels, fluorophores, chromophores, etc. Techniques for constructing arrays and methods of using these arrays are described in EPO 799 897; WO 97/29212; WO 97/27317; EP 0 785 280; WO 97/02357; U.S. Pat. No. 5,593,839; U.S. Pat. No. 5,578,832; EP 0 728 520; U.S. Pat. No. 5,599,695; EP 0 721 016; U.S. Pat. No. 5,556,752; WO 95/22058; and U.S. Pat. No. 5,631,734. Further, arrays can be used to examine differential expression of genes and can be used to determine gene function. For example, arrays of the instant polynucleotide sequences can be used to determine if any of the polynucleotide sequences are differentially expressed between normal cells and diseased cells. High expression of a particular message in a diseased sample, which is not observed in a corresponding normal sample, can indicate a cancer specific protein.

### (H) Data Analysis Methods

Comparison of the expression levels of one or more “CANCER GENES” with reference expression levels, e.g., expression levels in diseased cells of cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides a computer readable form of the gene expression profile data of the invention, or of values corresponding to the level of expression of at least one “CANCER GENE” in a diseased cell. The values can be mRNA expression levels obtained from experiments, e.g., microarray analysis. The values can also be mRNA levels normalized relative to a reference gene whose expression is constant in numerous cells under numerous conditions, e.g., GAPDH. In other embodiments, the values in the computer are ratios of, or differences between, normalized or non-normalized mRNA levels in different samples.

The gene expression profile data can be in the form of a table, such as an Excel table. The data can be alone, or it can be part of a larger database, e.g., comprising other expression profiles. For example, the expression profile data of the invention can be part of a public database. The computer readable form can be in a computer. In another embodiment, the invention provides a computer displaying the gene expression profile data.

In one embodiment, the invention provides a method for determining the similarity between the level of expression of one or more “CANCER GENES” in a first cell, e.g., a cell of a subject, and that in a second cell, comprising obtaining the level of expression of one or more “CANCER GENES” in a first cell and entering these values into a computer comprising a database including records comprising values corresponding to levels of expression of one or more “CANCER GENES” in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of “CANCER GENES” are entered into a computer system, comprising one or more databases with reference expression levels obtained from more than one cell. For example, the computer comprises expression data of diseased and normal cells. Instructions are provided to the computer, and the computer is capable of comparing the data entered with the data in the computer to determine whether the data entered is more similar to that of a normal cell or of a diseased cell. In another embodiment, the computer comprises values of expression levels in cells of subjects at different stages of cancer, and the computer is capable of comparing expression data entered into the computer with the data stored, and produces results indicating to which of the expression profiles in the computer, the one entered is most similar, such as to determine the stage of cancer in the subject.

In yet another embodiment, the reference expression profiles in the computer are expression profiles from cells of cancer of one or more subjects, which cells are treated in vivo or in vitro with a drug used for therapy of cancer. Upon entering of expression data of a cell of a subject treated in vitro or in vivo with the drug, the computer is instructed to compare the data entered to the data in the computer, and to provide results indicating whether the expression data entered into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides a system that comprises a means for receiving gene expression data for one or a plurality of genes; a means for comparing the gene expression data from each of said one or plurality of genes to a common reference frame; and a means for presenting the results of the comparison. This system may further comprise a means for clustering the data.

In another embodiment, the invention provides a computer program for analyzing gene expression data comprising (i) a computer code that receives input gene expression data for a plurality of genes and (ii) a computer code that compares said gene expression data from each of said plurality of genes to a common reference frame.

The invention also provides a machine-readable or computer-readable medium including program instructions for performing the following steps: (i) comparing a plurality of values corresponding to expression levels of one or more genes characteristic of cancer in a query cell with a database including records comprising reference expression or expression profile data of one or more reference cells and an annotation of the type of cell; and (ii) indicating to which cell the query cell is most similar based on similarities of expression profiles. The reference cells can be cells from subjects at different stages of cancer. The reference cells can also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides a method for selecting a therapy for a patient having cancer, the method comprising: (i) providing the level of expression of one or more genes characteristic of cancer in a diseased cell of the patient; (ii) providing a plurality of reference profiles, each associated with a therapy, wherein the subject expression profile and each reference profile has a plurality of values, each value representing the level of expression of a gene characteristic of cancer; and (iii) selecting the reference profile most similar to the subject expression profile, to thereby select a therapy for said patient. In a preferred embodiment step (iii) is performed by a computer. The most similar reference profile may be selected by weighing a comparison value of the plurality using a weight value associated with the corresponding expression data.

The relative abundance of a mRNA in two biological samples can be scored as a perturbation and its magnitude determined (i.e., the abundance is different in the two sources of mRNA tested), or as a non-perturbation (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations can be used by a computer for calculating and expressing comparisons.

Preferably, in addition to identifying a perturbation as positive or negative, it is advantageous to determine the magnitude of the perturbation. This can be carried out, as noted above, by calculating the ratio of the emission of the two fluorophores used for differential labeling, or by analogous methods that will be readily apparent to those of skill in the art.

The computer readable medium may further comprise a pointer to a descriptor of a stage of cancer or to a treatment for cancer.

In operation, the means for receiving gene expression data, the means for comparing gene expression data, the means for presenting, the means for normalizing, and the means for clustering within the context of the systems of the present invention can involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that can cause a computer to function in the particular fashion described herein.

Those skilled in the art will understand that the systems and methods of the present invention may be applied to a variety of systems, including IBM-compatible personal computers running MS-DOS or Microsoft Windows.

In an exemplary implementation, to practice the methods of the present invention, a user first loads expression profile data into the computer system. These data can be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

### (I) In Situ Hybridization

In one aspect, the method comprises in situ hybridization with a probe derived from a given marker polynucleotide, whose sequence is selected from any of the polynucleotide sequences of the genes listed in Table 1 or a sequence complementary thereto. The method comprises contacting the labeled hybridization probe with a sample of a given type of tissue from a patient potentially having malignant neoplasia and cancer in particular as well as normal tissue from a person with no malignant neoplasia, and determining whether the probe labels the tissue of the patient to a degree significantly different (e.g., by at least a factor of two, or at least a factor of five, or at least a factor of twenty, or at least a factor of fifty) than the degree to which normal tissue is labeled. In situ hybridization may be performed either to DNA in the nucleus of said cell in the tissue or to the mRNA in the cytoplasm to stain for transcriptional activity.

### (J) Polypeptide Detection

Polypeptides being encoded by a marker gene of the present invention may be detected by immunohistochemical assays, dot-blot assays, ELISA and the like.

### (K) Antibodies

Any type of antibody known in the art can be generated to bind specifically to an epitope of a “CANCER GENE” polypeptide. An antibody as used herein includes intact immunoglobulin molecules, as well as fragments thereof, such as Fab, F(ab)₂, and Fv, which are capable of binding an epitope of a “CANCER GENE” polypeptide. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to an epitope of a “CANCER GENE” polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formations between an immunogen and an antibody which specifically binds to the immunogen.

Typically, an antibody which specifically binds to a “CANCER GENE” polypeptide provides a detection signal at least 5-, 10-, or 20-fold higher than a detection signal provided with other proteins when used in an immunochemical assay. Preferably, antibodies which specifically bind to “CANCER GENE” polypeptides do not detect other proteins in immunochemical assays and can immunoprecipitate a “CANCER GENE” polypeptide from solution.

“CANCER GENE” polypeptides can be used to immunize a mammal, such as a mouse, rat, rabbit, guinea pig, monkey, or human, to produce polyclonal antibodies. If desired, a “CANCER GENE” polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a “CANCER GENE” polypeptide can be prepared using any technique which provides for the production of antibody molecules by continuous cell lines in culture. These techniques include, but are not limited to, the hybridoma technique, the human B cell hybridoma technique, and the EBV hybridoma technique [Kohler et al., Nature 256 (1985), 495-7).

In addition, techniques developed for the production of chimeric antibodies, the splicing of mouse antibody genes to human antibody genes to obtain a molecule with appropriate antigen specificity and biological activity, can be used [Takeda et al., Nature 314 (1985), 452-4). Monoclonal and other antibodies also can be humanized to prevent a patient from mounting an immune response against the antibody when it is used therapeutically. Such antibodies may be sufficiently similar in sequence to human antibodies to be used directly in therapy or may require alteration of a few key residues. Sequence differences between rodent and human antibodies can be minimized by replacing residues which differ from those in the human sequences by site directed mutagenesis of individual residues or by grating of entire complementarity determining regions. Alternatively, humanized antibodies can be produced using recombinant methods, as described in GB2188638B. Antibodies which specifically bind to a “CANCER GENE” polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies which specifically bind to “CANCER GENE” polypeptides. Antibodies with related specificity, but of distinct idiotypic composition, can be generated by chain shuffling from random combinatorial immunoglobulin libraries [Burton, PNAS USA 88 (1991), 11120-3).

Single-chain antibodies can also be constructed using a DNA amplification method, such as PCR, using hybridoma cDNA as a template [Thirion et al., Eur. J. Cancer Prev. 5 (1996), 507-11). Single-chain antibodies can be mono- or bispecific, and can be bivalent or tetravalent. Construction of tetravalent, bispecific single-chain antibodies is taught, for example, in Coloma & Morrison, Nat. Biotechnol. 15 (1997), 159-63. Construction of bivalent, bispecific single-chain antibodies is taught in Mallender & Voss, J. Biol. Chem. Xno9 (1994), 199-206.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology [Verhaar et al., Int. J. Cancer 61 (1995), 497-501).

Antibodies which specifically bind to “CANCER GENE” polypeptides can also be produced by inducing in vivo production in a lymphocyte population or by screening immunoglobulin libraries or panels of highly specific binding reagents as disclosed in the literature [Orlandi et al., PNAS 86 (1989), 3833-7).

Other types of antibodies can be constructed and used therapeutically in methods of the invention. For example, chimeric antibodies can be constructed as disclosed in WO 93/03151. Binding proteins which are derived from immunoglobulins and which are multivalent and multispecific, such as the antibodies described in WO 94/13804, can also be prepared.

Antibodies according to the invention can be purified by methods well known in the art. For example, antibodies can be affinity purified by passage over a column to which a “CANCER GENE” polypeptide is bound. The bound antibodies can then be eluted from the column using a buffer with a high salt concentration.

Immunoassays are commonly used to quantify the levels of proteins in cell samples, and many such immunoassay techniques are known in the art. The invention is not limited to a particular assay procedure, and therefore is intended to include both homogeneous and heterogeneous procedures. Exemplary immunoassays which can be conducted according to the invention include fluorescence polarisation immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

Other methods of quantifying the level of a particular protein, or a protein fragment, or modified protein in a particular sample are based on flow-cytometric methods. Flow cytometry allows the identification of proteins on the cell surface as well as intracellular proteins using fluorochrome labeled, protein specific antibodies or non-labeled antibodies in combination with fluorochrome labeled secondary antibodies. General techniques to be used in performing flow cytometric assays noted above are known to those of ordinary skill in the art. A special method based on the same principles is the microsphere-based flow cytometry. Microsphere beads are labeled with precise quantities of fluorescent dye and specific antibodies. Such techniques are provided by WO 97/14028. In another embodiment the level of a particular protein or a protein fragment, or modified protein in a particular sample may be determined by 2D gel-electrophoresis and/or mass spectrometry. Determination of the protein nature, sequence, molecular mass as well charge can be achieved in one detection step. Mass spectrometry can be performed with methods known to those with skills in the art, such as MALDI, TOF, or combinations of these.

In another embodiment, the level of the encoded product, i.e., the product encoded by any of the polynucleotide sequences of the genes listed in Table 1 or a sequence complementary thereto, in a biological fluid (e.g., blood or urine) of a patient may be determined as a way of monitoring the level of expression of the marker polynucleotide sequence in cells of that patient. Such a method would include the steps of obtaining a sample of a biological fluid from the patient, contacting the sample (or proteins from the sample) with an antibody specific for a encoded marker polypeptide, and determining the amount of immune complex formation by the antibody, with such amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

In another embodiment, the method can be used to determine the amount of marker polypeptides present in a cell, which in turn can be correlated with progression of the disorder, e.g., plaque formation. The level of the marker polypeptides can be used predictively to evaluate whether a sample of cells contains cells which are, or are predisposed towards becoming, plaque associated cells. The evaluation of marker polypeptide levels can then be utilized in decisions regarding, e.g., the use of more stringent therapies.

As set out above, one aspect of the present invention relates to diagnostic assays for determining, in the context of cells isolated from a patient, if the level of a marker polypeptide is significantly reduced in the sample cells. The term “significantly reduced” refers to a cell phenotype wherein the cell possesses a reduced cellular amount of the marker polypeptide relative to a normal cell of similar tissue origin. For example, a cell may have less than about 50%, 25%, 10%, or 5% of the marker polypeptide that a normal control cell. In particular, the assay evaluates the level of the marker polypeptide in the test cells, and, preferably, compares this level with the level of the marker polypeptide detected in at least one control cell, e.g., a normal cell and/or a transformed cell of known phenotype.

Of particular importance to the subject invention is the ability to quantify the level of the marker polypeptide as determined by the number of cells associated with a normal or abnormal marker polypeptide level. The number of cells with a particular marker polypeptide phenotype may then be correlated with patient prognosis. In one embodiment of the invention, the marker polypeptide phenotype of the lesion is determined as a percentage of cells in a biopsy which are found to have abnormally high/low levels of the marker polypeptide. Such expression may be detected by immunohistochemical assays, dot-blot assays, ELISA and the like.

### (L) Immunohistochemistry

Where tissue samples are employed, immunohistochemical staining may be used to determine the number of cells having the marker polypeptide phenotype. For such staining, a multiblock of tissue is taken from the biopsy or other tissue sample and subjected to proteolytic hydrolysis, employing such agents as protease K or pepsin. In certain embodiments, it may be desirable to isolate the nuclear fraction from the sample cells and detect the level of the marker polypeptide in the nuclear fraction.

The tissues samples are fixed by treatment with a reagent such as formalin, glutaraldehyde, methanol, or the like. The samples are then incubated with an antibody, preferably a monoclonal antibody, with binding specificity for the marker polypeptide. This antibody may be conjugated to a Label for subsequent detection of binding. samples are incubated for a time sufficient for the formation of immunocomplexes. Binding of the antibody is then detected by virtue of a Label conjugated to this antibody. Where the antibody is unlabelled, a second labeled antibody may be employed, e.g., an antibody specific for the isotype of the anti-marker polypeptide antibody. Examples of labels which may be employed include radionuclide, fluorescence, chemoluminescence, and enzyme labels.

Where enzymes are employed, the substrate for the enzyme may be added to the samples to provide a colored or fluorescent product. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art.

In one embodiment, the assay is performed as a dot blot assay. The dot blot assay finds particular applications where tissue samples are employed, as it allows determination of the average amount of the marker polypeptide associated with a Single cell by correlating the amount of marker polypeptide in a cell-free extract produced from a predetermined number of cells.

In yet another embodiment, the invention contemplates using a panel of antibodies which are generated against the marker polypeptides of this invention, which polypeptides are encoded by any of the polynucleotide sequences of the genes from Table 1. Such a panel of antibodies may be used as a reliable diagnostic probe for cancer. The assay of the present invention comprises contacting a biopsy sample containing cells, e.g., macrophages, with a panel of antibodies to one or more of the encoded products to determine the presence or absence of the marker polypeptides. The diagnostic methods of the subject invention may also be employed as follow-up to treatment, e.g., quantification of the level of marker polypeptides may be indicative of the effectiveness of current or previously employed therapies for malignant neoplasia and cancer in particular as well as the effect of these therapies upon patient prognosis.

The diagnostic assays described above can be adapted to be used as prognostic assays, as well. Such an application takes advantage of the sensitivity of the assays of the Invention to events which take place at characteristic stages in the progression of plaque generation in case of malignant neoplasia. For example, a given marker gene may be up- or down-regulated at a very early stage, perhaps before the cell develops into a foam cell, while another marker gene may be characteristically up or down regulated only at a much later stage. Such a method could involve the steps of contacting the mRNA of a test cell with a polynucleotide probe derived from a given marker polynucleotide which is expressed at different characteristic levels in cancer tissue cells at different stages of malignant neoplasia progression, and determining the approximate amount of hybridization of the probe to the mRNA of the cell, such amount being an indication of the level of expression of the gene in the cell, and thus an indication of the stage of disease progression of the cell; alternatively, the assay can be carried out with an antibody specific for the gene product of the given marker polynucleotide, contacted with the proteins of the test cell. A battery of such tests will disclose not only the existence of a certain neoplastic lesion, but also will allow the clinician to select the mode of treatment most appropriate for the disease, and to predict the likelihood of success of that treatment.

The methods of the invention can also be used to follow the clinical course of a given cancer predisposition. For example, the assay of the Invention can be applied to a blood sample from a patient; following treatment of the cancer patient, another blood sample will be taken and the test repeated. Successful treatment may result in removal of the demonstrated differential expression, characteristic of the cancer tissue cells, perhaps approaching normal levels.

### (M) Modulation of Gene Expression

Test compounds which increase or decrease “CANCER GENE” expression can be identified. A “CANCER GENE” polynucleotide is contacted with a test compound in an appropriate expression test system as described below or in a cell system, and the expression of an RNA or polypeptide product of the “CANCER GENE” polynucleotide is determined. The level of expression of the appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression.

The level of “CANCER GENE” mRNA or polypeptide expression in the cells can be determined by methods well known in the art for detecting mRNA or polypeptide, e.g., as described above. Either qualitative or quantitative methods can be used. Alternatively, polypeptide synthesis can be determined in vivo, in a cell culture, or in an in vitro translation system by detecting incorporation of labeled amino acids into a “CANCER GENE” polypeptide. Such screening can be carried out either in a cell-free assay system or in an intact cell. Any cell which expresses a “CANCER GENE” polynucleotide can be used in a cell-based assay system. A “CANCER GENE” polynucleotide can be naturally occurring in the cell or can be introduced using techniques such as those described above. Either a primary culture or an established cell line, such as CHO or human embryonic kidney 293 cells, can be used.

One strategy for identifying genes that are involved in cancer is to detect genes that are expressed differentially under conditions associated with the disease versus non-disease or in the context of therapy response conditions. The sub-sections below describe a number of experimental systems which can be used to detect such differentially expressed genes. In general, these experimental systems include at least one experimental condition in which subjects or samples are treated in a manner associated with cancer, in addition to at least one experimental control condition lacking such disease associated treatment or lacking a response to such treatment. Differentially expressed genes are detected, as described below, by comparing the pattern of gene expression between the experimental and control conditions.

Once a particular gene has been identified through the use of one such experiment, its expression pattern may be further characterized by studying its expression in a different experiment and the findings may be validated by an independent technique. Such use of multiple experiments may be useful in distinguishing the roles and relative importance of particular genes in cancer and the treatment thereof. A combined approach, comparing gene expression pattern in cells derived from cancer patients to those of in vitro cell culture models can provide substantial information on the pathways involved in development and/or progression of cancer. It can also elucidate the role of such genes in the development of resistance or insensitivity to certain therapeutic agents (e.g. chemotherapeutic drugs).

Among the experiments which may be utilized for the identification of differentially expressed genes involved in malignant neoplasia and cancer in particular, are experiments designed to analyze those genes which are involved in signal transduction. Such experiments may serve to identify genes involved in the proliferation of cells.

Below are methods described for the identification of genes which are involved in cancer. Such representative genes may be differentially expressed in cancerous conditions relative to their expression in normal, or non-cancerous conditions or upon experimental manipulation based on clinical observations. Such differentially expressed genes represent “target” and/or “marker” genes. Methods for further characterization of such differentially expressed genes, and for their identification as target and/or marker genes, are presented below.

Alternatively, a differentially expressed gene may have its expression modulated, i.e., quantitatively increased or decreased, in normal versus cancerous states, or under controlled versus experimental conditions. The degree to which expression differs in normal versus cancerous or control versus experimental states need only be large enough to be visualized via standard characterization techniques, such as, for example, the differential display technique described below. Other such standard characterization techniques by which expression differences may be visualized include but are not limited to quantitative RT-PCR and Northern analyses, which are well known to those of skill in the art.

In addition to the experiments described above the following describes algorithms and statistical analyses which can be utilized for data evaluation and for the classification, as well as, response prediction for a so far not classified biological sample in the context of control samples. The predictive algorithms and equations described below have already shown their power to subdivide individual cancers.

### EXAMPLE 2

### Expression Profiling Utilizing Quantitative Kinetic RT-PCR

Using the PRISM 7700 or 7900 Sequence Detection System of PE Applied Biosystems (Perkin Elmer, Foster City, Calif., USA) with the technique of a fluorogenic probe, consisting of an oligonucleotide labeled with both a fluorescent reporter dye and a quencher dye, expression measurement was performed. Amplification of the probe-specific product causes cleavage of the probe, generating an increase in reporter fluorescence. Primers and probes were selected using the Primer Express software and localized mostly across exon/intron borders and large intervening non-transcriped sequences (>800 bp) to guarantee RNA-specificity or within the 3′ region of the coding sequence or in the 3′ untranslated region. Predefined primer and probes for the genes listed in Table 1 can also be obtained from suppiers e.g. PE Applied Biosystems. All primer pairs were checked for specificity by conventional PCR reactions and gel electrophoresis. To standardize the amount of sample RNA, GAPDH, RPL37A, RPL9 and CD63 were selected as references, since they were not differentially regulated in the samples analyzed. To perform such an expression analysis of genes within a biological samples the respective primer/probes were prepared by mixing 25 μl of the 100 μM stock solution “Upper Primer”, 25 μl of the 100 μM stock solution “Lower Primer” with 12.5 μl of the 100 μM stock solution TaqMan-probe (FAM/Tamra) and adjusted to 500 μl with aqua dest (Primer/probe-mix). For each reaction 1.25 μl cDNA of the patient samples were mixed with 8.75 μl nuclease-free water and added to one well of a 96 Well-Optical Reaction Plate (Applied Biosystems Part No. 4306737). 1.5 μl of the Primer/Probe-mix described above, 12.5 μl Taq Man Universal-PCR-mix (2×) (Applied Biosystems Part No. 4318157) and 1 μl water were then added. The 96 well plates were closed with 8 Caps/Strips (Applied Biosystems Part Number 4323032) and centrifuged for 3 minutes. Measurements of the PCR reaction were done according to the instructions of the manufacturer with a TaqMan 7700 from Applied Biosystems (No. 20114) under appropriate conditions (2 min. 50° C., 10 min. 95° C., 0.15 min. 95° C., 1 min. 60° C.; 40 cycles). Prior to the measurement of so far unclassified biological samples control experiments with e.g. cell lines, healthy control samples, samples of defined therapy response were used for standardization of the experimental conditions.

TaqMan validation experiments were performed showing that the efficiencies of the target and the control amplifications are approximately equal which is a prerequisite for the relative quantification of gene expression by the comparative ΔΔCT method, known to those with skills in the art. The SoftwareSDS 2.0 from Applied Biosystems was used according to the respective instructions. CT-values were then further analyzed with appropriate software (Microsoft Excel™) of statistical software packages (SAS).

TABLE 1
Genes differentially expressed and capable of
predicting therapeutic success.

| | Gene_Symbol | Ref. Sequences | Locus_Link_ID | Unigene_ID | OMIM |
|---|---|---|---|---|---|
| SEQ NO | [A] | [A] | [A] | [A] | [A] |
| 1 | VEGFC | NM_005429 | 7424 | 79141 | 601528 |
| 2 | EGFR | NM_005228 | 1956 | 77432 | 131550 |
| 3 | ERBB2 | NM_004448 | 2064 | 323910 | 164870 |
| | Her-2neu | | | | |
| 4 | ERBB3 | NM_001982 | 2065 | 199067 | 190151 |
| 5 | ERBB4 | NM_005235 | 2066 | 1939 | 600543 |
| 6 | KRT5 | NM_000424 | 3852 | 195850 | 148040 |
| 7 | KRT14 | NM_000526 | 3861 | 117729 | 148066 |
| 8 | FLT3 | NM_004119 | 2322 | 385 | 136351 |
| 9 | FLT4 | NM_002020 | 2324 | 74049 | 136352 |
| 10 | KDR | NM_002253 | 3791 | 12337 | 191306 |
| 11 | VEGFA | NM_003376 | 7422 | 73793 | 192240 |
| 12 | VEGFB | NM_003377 | 7423 | 78781 | 601398 |

### EXAMPLE 3

### Expression Profiling Utilizing DNA Microarrays

Expression profiling was carried out using the Affymetrix Array Technology. By hybridization of mRNA to such a DNA-array or DNA-Chip, it was possible to identify the expression value of each transcripts due to signal intensity at certain position of the array. Usually these DNA-arrays are produced by spotting of cDNA, oligonucleotides or subcloned DNA fragments. In case of Affymetrix technology app. 400.000 individual oligonucleotide sequences were synthesized on the surface of a silicon wafer at distinct positions. The minimal length of oligomers is 12 nucleotides, preferable 25 nucleotides or full length of the questioned transcript. To determine the quantitative and qualitative changes in the gene expression of certain cancer specimens, RNA from tumor tissue extracted prior to any chemotherapy was compared among each other individually and/or to RNA extracted from benign tissue (e.g. epithelial tissue, or micro dissected ductal tissue) on the basis of expression profiles for the whole transcriptome. With minor modifications, the sample preparation protocol followed the Affymetrix GeneChip Expression Analysis Manual (Santa Clara, Calif.). Total RNA extraction and isolation from tumor or benign tissues, biopsies, cell isolates or cell containing body fluids was performed by using TRIzol (Life Technologies, Rockville, Md.) and Oligotex mRNA Midi kit (Qiagen, Hilden, Germany). An ethanol precipitation step was carried out to bring the concentration to 1 mg/ml. 5-10 mg of mRNA were used to create double stranded cDNA by the SuperScript system (Life Technologies). First strand cDNA synthesis was primed with a T7-(dT24) oligonucleotide. The cDNA was extracted with phenol/chloroform and precipitated with ethanol to a final concentration of 1 mg/ml. From the generated cDNA, cRNA was synthesized using Enzo's (Enzo Diagnostics Inc., Farmingdale, N.Y.) in vitro Transcription Kit. Within the same step the cRNA was labeled with biotin nucleotides Bio-11-CTP and Bio-16-UTP (Enzo Diagnostics Inc., Farmingdale, N.Y.). After labeling and cleanup (Qiagen, Hilden (Germany) the cRNA then was fragmented in an appropriated fragmentation buffer (40 mM Tris-Acetate, pH 8.1, 100 mM KOAc, 30 mM MgOAc, for 35 minutes at 94° C.). As per the Affymetrix protocol, fragmented cRNA were hybridized on the HG_U133 arrays comprising app. 40.000 probed transcripts each, for 24 hours at 60 rpm in a 45° C. hybridization oven. After the hybridization step the chip surfaces were washed and stained with streptavidin phycoerythrin (SAPE; Molecular Probes, Eugene, Oreg.) in Affymetrix fluidics stations. To amplify staining, a second labeling step was introduced, which is recommended but not compulsive. SAPE solution was added twice with an antistreptavidin biotinylated antibody. Hybridization to the probe arrays was detected by fluorometric scanning (Hewlett Packard Gene Array Scanner; Hewlett Packard Corporation, Palo Alto, Calif.).

After hybridization and scanning, the microarray images were analyzed for quality control, looking for major chip defects or abnormalities in hybridization signal. Therefore, Affymetrix GeneChip MAS 5.0 Software was utilized. Primary data analysis was carried out by software provided by the manufacturer. The primary data have been analyzed by further bioinformatic tools and additional filter criteria as described in Example 4.

### EXAMPLE 4

### Data Analysis from Expression Profiling Experiments

According to Affymetrix measurement technique (Affymetrix GeneChip Expression Analysis Manual, Santa Clara, Calif.) a single gene expression measurement on one chip yielded the average difference value and the absolute call. Each chip contains 16-20 oligonucleotide probe pairs per gene or cDNA clone. These probe pairs include perfectly matched sets and mismatched sets, both of which are necessary for the calculation of the average difference, or expression value, a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. The average difference is a numeric value supposed to represent the expression value of that gene. The absolute call can take the values ‘A’ (absent), ‘M’ (marginal), or ‘P’ (present) and denotes the quality of a single hybridization. In the present experiment, both the quantitative information given by the average difference and the qualitative information given by the absolute call were used to identify the genes which are differentially expressed in biological samples from individuals with cancer versus biological samples from the normal population. With other algorithms than the Affymetrix one different numerical values representing the same expression values and expression differences upon comparison were obtained.

The differential expression E in one of the cancer groups compared to the normal population was calculated as follows. Given n average difference values d1, d2, . . . , dn in the cancer population and m average difference values c1, c2, . . . , cm in the population of normal individuals, it is computed by the equation:

$$E ≡ exp ( \frac{1}{m} {\sum }_{i = 1}^{m} ln ( {c}_{i} ) - \frac{1}{n} {\sum }_{i = 1}^{n} ln ( {d}_{i} ) ) \qquad ( equation 1 )$$

If dj<50 or ci<50 for one or more values of i and j, these particular values ci and/or dj were set to an “artificial” expression value of 50. These particular computations of E allowed for a correct comparison to TaqMan results.

A gene was called up-regulated in cancer of good or bad outcome, if E>=average change factor given in Table 2 and if the number of absolute calls equaled to ‘P’ in the cancer population was greater than n/2. The average fold change factors in Table 2 were given for those patients suffering a tumor resulting in an overall survival time of less than 27 month (sample group 1), and those suffering a tumor resulting in an overall survival time of more than 50 month (sample group 2) or those patients suffering a tumor with an overall survival time of at least 27 month up to now (sample group 3).

TABLE 2
Fold Change values of genes differentially expressed
and capable of predicting therapeutic success based
on 3 independent qRT-PCR experimental runs (I-III).

| | Gene | |
|---|---|---|
| Gene Name | Description | Score |
| Gapdh I | normalized to RPL37A | 1.595 |
| RPL37A I | normalized to RPL37A | 1 |
| KRT5 I | normalized to RPL37A | 2.084 |
| KRT14 I | normalized to RPL37A | 1.339 |
| V1-ex4 I | normalized to RPL37A | 1.208 |
| V189 I | normalized to RPL37A | 2.88 |
| V165 I | normalized to RPL37A | 1.263 |
| V121 I | normalized to RPL37A | 2.328 |
| V2-ex8 I | normalized to RPL37A | 1.355 |
| EGFR I | normalized to RPL37A | 1.078 |
| VEGFC I | normalized to RPL37A | 1.592 |
| Her2/neu I | normalized to RPL37A | 1.842 |
| ERBB4 I | normalized to RPL37A | 4.454 |
| RPL9 I | normalized to RPL37A | 1.126 |
| CD63 I | normalized to RPL37A | 1.477 |
| Gapdh II | normalized to RPL37A | 1.573 |
| RPL37A II | normalized to RPL37A | 1 |
| ERBB3 II | normalized to RPL37A | 2.061 |
| ERBB4 II | normalized to RPL37A | 1.064 |
| V1-ex8 II | normalized to RPL37A | 1.223 |
| V1 + V189 II | normalized to RPL37A | 1.502 |
| V1 + V165 II | normalized to RPL37A | 1.181 |
| V1 + V121 II | normalized to RPL37A | 1.239 |
| VEGF-B II | normalized to RPL37A | 1.193 |
| VEGFC II | normalized to RPL37A | 7.438 |
| EGF-R II | normalized to RPL37A | 1.76 |
| KDR II | normalized to RPL37A | 1.398 |
| FLT1 II | normalized to RPL37A | 1.17 |
| FLT4 II | normalized to RPL37A | 1.038 |
| RPL9 II | normalized to RPL37A | 1.281 |
| CD63 II | normalized to RPL37A | 1.132 |
| Gapdh III | normalized to RPL37A | 1.105 |
| RPL37A III | normalized to RPL37A | 1 |
| EGF-R III | normalized to RPL37A | 1.104 |
| Her2/neu III | normalized to RPL37A | 2.004 |
| ERBB3 III | normalized to RPL37A | 1.821 |
| ERBB4 III | normalized to RPL37A | 1.147 |
| KRT5 III | normalized to RPL37A | 2.916 |
| KRT14 III | normalized to RPL37A | 4.497 |
| V1-ex4 III | normalized to RPL37A | 1.242 |
| VEGF-B III | normalized to RPL37A | 1.299 |
| VEGFC III | normalized to RPL37A | 7.378 |
| KDR III | normalized to RPL37A | 1.122 |
| FLT1 III | normalized to RPL37A | 1.182 |
| FLT4-1 III | normalized to RPL37A | 1.291 |
| RPL9 III | normalized to RPL37A | 1.357 |
| CD63 III | normalized to RPL37A | 1.483 |

Fold changes greater than 1 refer to a difference in gene expression between the first and second sample cohort. This regulation factors are mean values and may differ individually, here the combined profiles of all 12 genes listed in Table 1 in a cluster analysis or a principle component analysis (PCA) indicate the classification group for such sample (See FIG. 4 for representative PCA with 3 genes and two classes). By a PCA one will identify the major components (Eigengenes or Eigenvectors) which do discriminate the samples analyzed.

### Data Filtering

Raw data of the qRT-PCR were normalized to one or combinations of the housekeeping genes RPL37A, GAPDH, RPL9 and CD63 by using the comparative ΔΔCT method, known to those skilled in the art. In brief, all experiments were normalized by adjusting the respective housekeeping gene to a CT value of 25. “Copy numbers” of each gene were then calculated by 2^{(40-gene×normalized CT value)}. Raw data of gene array analysis were acquired using Microsuite 5.0 software of Affymetrix and normalized following a standard practice of scaling the average of all gene signal intensities to a common arbitrary value. 59 Genes corresponding to Affymetrix controls (housekeeping genes, etc.) were removed from the analysis. The only exception has been done for the genes for GAPDH and Beta-actin, which expression levels were used for the normalization purposes. One hundred genes, which expression levels are routinely used in order to normalize between HG-U133A and HG-U133B GeneChips, were also removed from the analysis. Genes with potentially high levels of noise (81 probe sets), which is observed for genes with low absolute expression values (genes, which expression levels did not achieve 30 RLU (TGT=100) through all experiments), were removed from the data set. The remaining genes were preprocessed to eliminate the genes (3196 probe sets) whose signal intensities were not significantly different from their background levels and thus labeled as “Absent” by Affymetrix MicroSuite 5.0 in all experiments. Genes were eliminated that were not present in at least 10% of samples (3841 probe sets). Data for remaining 15,006 probe sets were subsequently analyzed by statistical methods.

### Statistical Analysis

In order to optimize prediction of outcome this class from the training cohort was used and multiple statistical tests were carried out, suitable for group comparison including nonparametric Wilcoxon rank sum test, two-sample independent Students' t-test, Welch test, Kolmogorov-Smirnov test (for variance), and SUM-Rank test (see Table 3). As shown, such genes with a differential expression in the metastasis group vs. the non metastasis group and a significance level (p-value) below 0.05 could be identified. Hereby statistical significance of the selected candidate genes displayed in Table 1 was verified.

TABLE 3
p-values for statistical significance of genes
predicting overall survival of HNSCC patients

| | | | Kolmogorov- | | Rank |
|---|---|---|---|---|---|
| Gene Name | T-Test | Welch | Smirnov | Wilcoxon | Sum |
| ERBB4 | 0.0104 | 0.0035 | 0.0606 | 0.0167 | 1 |
| VEGFC II | 0.0186 | 0.0228 | 0.0476 | 0.0822 | 2 |
| KRT5 | 0.0203 | 0.0177 | 0.0664 | 0.0160 | 3 |
| VEGFC | 0.0374 | 0.0209 | 0.0606 | 0.0553 | 4 |
| Her2/neu | 0.0786 | 0.0510 | 0.0190 | 0.0559 | 5 |
| VEGF-B II | 0.1455 | 0.1061 | 0.0657 | 0.1490 | 6 |
| V1 + V189 | 0.2054 | 0.1647 | 0.0657 | 0.1490 | 7 |
| II | | | | | |
| ERBB3 II | 0.2159 | 0.1884 | 0.1392 | 0.2824 | 8 |
| KRT14 | 0.4276 | 0.3761 | 0.0922 | 0.1471 | 9 |
| V1-ex4 | 0.4200 | 0.3536 | 0.3049 | 0.3132 | 10 |
| KDR II | 0.2598 | 0.1959 | 0.6838 | 0.5237 | 11 |
| V2-ex8 | 0.4837 | 0.5254 | 0.5131 | 0.4606 | 12 |
| EGF-R | 0.4435 | 0.3967 | 0.8354 | 0.6354 | 13 |
| V189 | 0.5049 | 0.5189 | 0.7077 | 0.6042 | 14 |
| EGF-R II | 0.4684 | 0.4224 | 0.9520 | 0.7546 | 15 |
| V1-ex8 II | 0.8362 | 0.8250 | 0.4602 | 0.4908 | 16 |
| V165 | 0.6479 | 0.6131 | 0.7912 | 0.7972 | 17 |
| ERBB4 II | 0.5798 | 0.5465 | 0.9091 | 1.0000 | 18 |
| V1 + V165 | 0.7077 | 0.6839 | 0.9627 | 0.7242 | 19 |
| II | | | | | |
| V1 + V121 II | 0.8208 | 0.8152 | 0.6374 | 0.8518 | 20 |
| V121 | 0.6573 | 0.6537 | 0.9997 | 0.7679 | 21 |
| FLT4-1 II | 0.9396 | 0.9474 | 0.7692 | 0.8329 | 22 |

### EXAMPLE 5

### Statistical Relevance of Candidate Genes Differentially Expressed in Cancers for Overall Survival Discrimination

While those algorithms described in Example 4 can be implemented in a certain kernel to classify samples according to their specific gene expression into two classes another approach can be taken to predict class membership by implementation of a k-NN classification. The method of k-Nearest Neighbors (k-NN), an important approach to nonparametric classification, is quite easy and efficient. Partly because of its perfect mathematical theory, NN method develops into several variations. As we know, if we have infinitely many sample points, then the density estimates converge to the actual density function. The classifier becomes the Bayesian classifier if the large-scale sample is provided. But in practice, given a small sample, the Bayesian classifier usually fails in the estimation of the Bayes error especially in a high-dimensional space, which is called the disaster of dimension. Therefore, the method of k-NN has a great pity that the sample space must be large enough.

In k-nearest-neighbor classification, the training data set was used to classify each member of a “target” data set. The structure of the data is that there is a classification (categorical) variable of interest (e.g. “long-term survivors” (sample group 2) or “short-term survivors” (sample group 1)), and a number of additional predictor variables (gene expression values). Generally speaking, the algorithm is as follows:

1. For each sample in the data set to be classified, locate the k nearest neighbors of the training data set. A Euclidean distance measure or a correlation analysis can be used to calculate how close each member of the training set is to the target sample that is being examined.
2. Examine the k nearest neighbors—which classification do most of them belong to?
3. Assign this category to the sample being examined.
4. Repeat procedure steps 1 to 3 for the remaining samples in the target set.

Of course the computing time goes up as k goes up, but the advantage is that higher values of k provide smoothing that reduces vulnerability to noise in the training data. In practical applications, typically, k is in units or tens rather than in hundreds or thousands. In this experiment a k=3 was used.

The “nearest neighbors” are determined if given the considered the vector and the distance measurement. Given a training set of expression values for a certain number of samples

T={(x1, y1), (x2, y2), . . . , (xm, ym)}, to determine the class of the input vector x.

The most special case is the k-NN method, while k=1, which just searches the one nearest neighbor:

j=argmin//x−xi//

then, (x, yj) is the solution.

For estimation on the error rate of this classification the following considerations could be made:

A training set T={(x1, y1), (x2, y2), . . . , (xm, ym)} is called (k, d %)-stable if the error rate of k-NN method is d %, where d % is the empirical error rate from independent experiments. If the clustering of data are quite distinct (the class distance is the crucial standard of classification), then the k must be small. The key idea is the least k in the case that d % is bigger than the threshold value is preferred.

The k-NN method gathers the nearest k neighbors and let them vote—the class of most neighbors wins. Theoretically, the more neighbors one considers, the smaller error rate it takes place. The general case is a little more complex. But by imagination, it is true to be the more k the lower upper bound asymptotic to PBayes(e) if N is fixed.

One can use such algorithm to classify and cross validate a given cohort of samples based on the genes presented by this invention in Table 1. Most preferably the classification shall be performed based on the expression levels of the genes presented in Table 1 but may also be combined with clinicopathological data as far a they are measured in a continous manner (e.g. immunehistochemistry data, scoring data such as TNM status or biochemical properties of such tumor tissue.

With k=3 and >100 iteration one can get classifications as depicted below for a cross-validation experiment with the two classes “long-term survivors” (sample group 2) or “short-term survivors”.

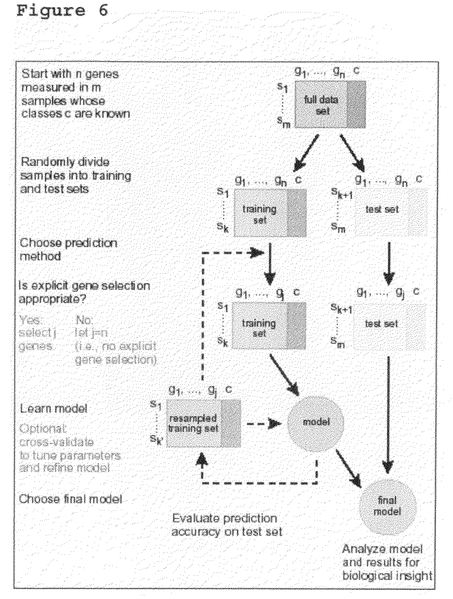

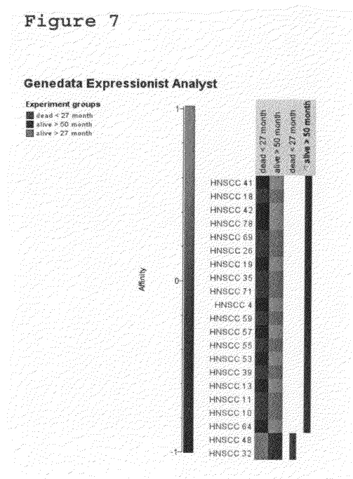

The misclassification of some samples or not classifiable samples may be due to low tumor amount in specimen. The process of model generation and cross-validation of predictive gene sets may follow the path outlined in FIG. 6, wherein a given cohort of samples is subdivided into two sets a so called training and a test set. Based on such training set genes can be picked and a preliminary model can be evaluated, further such model can be validated with the sample taken from the test set cohort. These two independent classifications of samples will lead to a final model (e.g. KNN algorithm and matrix) which can be further applied to new independent tumor samples.

### EXAMPLE 6

### Prognosis/Prediction for Overall Survival of Cancer Patients Based on the Expression Levels of Genes Listed in Table 1

In order to get the most accurate prognosis/prediction for overall survival of cancer patients based on the expression levels of genes listed in Table 1. A step wise classification model (e.g. decision tree) identifying first those individuals (tumor tissues) with the highest affinity (e.g. by k-NN classification) to the class of long term survivors tumors (good prognosis group, alive>50 month) was implemented. If a so far unclassified tumor sample did not belong to this class on may perform a second classification step for this sample using the expression levels of the genes from Table 1 and some of the established clinicopathological parameters such as TNM classification. Nevertheless a classification by the genes listed in Table 1 is sufficient to identify patients not being at risk for early death or those who should receive additional treatment (e.g. Avastin, Iressa, Sorafenib, SU 11248) as being at high risk of early death (within first 27 month).

### EXAMPLE 7

### Correlation of Overall Survival on Basis of Candidate Gene Expression

To correlate overall survival on the basis of candidate gene expression Kaplan-Meier calculations were performed. Kaplan-Meier calculations are very well known to those skilled in the art. Graphpad Prism™ 4 was used. Overall survival data were censored and correlated to the gene expression levels.

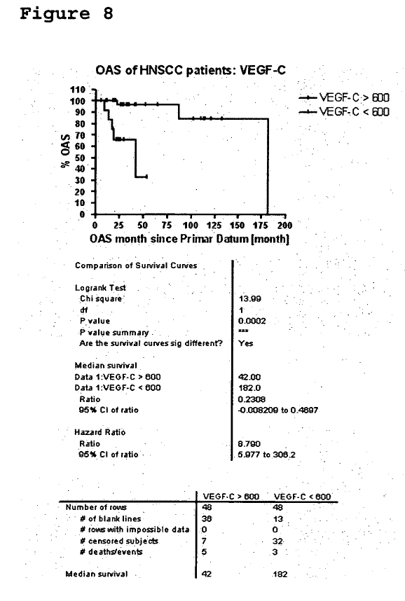

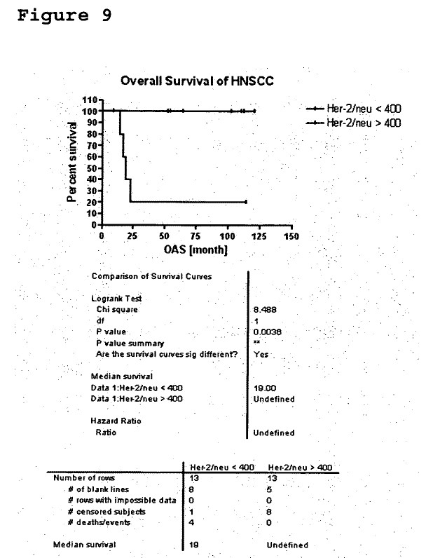

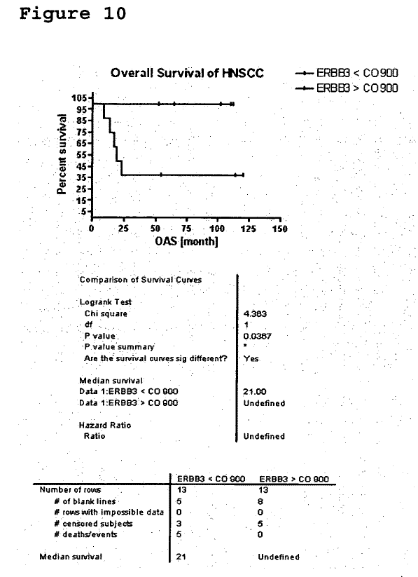

An example of such an analysis is given in FIG. 8. FIGS. 9 and 10 show the overall survival proportion in respect to gene expression of candidate genes (VEGFC, Her-2/neu and ERBB3). We have found that the determination of elevated VEGFC expression correlates with bad outcome of patients (see also FIG. 2). VEGFC preferably binds to VEGFR3 (=FLT4), but also VEGFR2 (=KDR). VEGFR3 is predominantly expressed on lymphatic vessels in the adult organism. Therefore, the overexpression of VEGFC results in the attraction and subsequent recruitment of lymphatic vessels into proximity to the tumor. This may ultimately lead to establishment of intratumoral lymphatic vessels thereby facilitating and enhancing the dissemination of tumor cells into lymphnodes and formation of distant metastasis. We conclude, that VEGFC is sufficient to do prediction/prognosis of cancer, that is metastasizing via the lymphatic vessel system as exemplified for HNSCC. Cancer patients whose tumors exhibit elevated VEGFC expression do particular benefit from treatments blocking the VEGF-VEGFR system. In particular elevated VEGFC expression indicates sensitivity towards small molecule inhibitors targeting the VEGFR, such as Sorafenib (BAY 43-9006), BAY 43-9005, BAY 57-9352, Sutent (SU11248), SU6668, Iressa, AZD6474, AZD2171, AZD6126, PTK787-ZK222584, CP547632, GW786034, CEP7055. In addition VEGFC also indicates patients being sensitive for anti-VEGF antibodies. Preferably these antibodies should be targeted to VEGFC. However, VEGFC also generally indicates vascularization, which is at least to some extent assisted by VEGF alpha isoforms. Therefore anti-cancer strategies using antibodies binding to all or singular isoforms of VEGF alpha, such as VEGF alpha 121, 145, 165, 189, 206, are also particularly effective in VEGFC expressing tumors. In addition VEGFC expression and simultaneous expression of respective receptors by the surrounding tumor mesenchyme or the tumor cells itself enables paracrine and autocrine growth and survival mechanisms. Therefore not only the recruitemnet of lymphatic vessels is important with respect to VEGFC expression. This enable the possibility that therapeutics raised against the VEGF-VEGFR system (as depicted above) also directly or more directly affect the tumor cells itself.

We have found that the measurement of solely VEGFC is sufficient to do prognosis of cancer and prediction of tumor response to anti-tumor treatment, that is metastasizing via the lymphatic vessel system.

Therefore a method of any one of claims 1 to 3, comprising
(a) obtaining a biological sample from a patient;
(b) determining the pattern of expression levels of VEGFC;
(c) comparing the pattern of expression levels determined in (b) with one or several reference pattern(s) of expression levels;
   wherein (i) upregulated expression of VEGFC is indicative of a poor prognosis as regards therapeutic success for said given mode of treatment in said subject is useful. In particular to select for anti VEGF-VEGFR regimens to treat the patients.

In addition, we have found that the expression of members of the EGFR family is critical for survival of patients. The composition of the EGFR family members affects the downstream signaling. It seems that expression of EGFR in the relative absence of other EGFR family members, i.e. ERBB2, ERBB3 and ERBBB4, is unfavorable. We conclude that the expression of EGFR family members affect the VEGF ligand expression. In particular, we conclude that EGFR expression positively influences VEGFC expression. Coexpression of other family members like ERBB2 and ERBB3 might negatively influence VEGFC expression.

As a reduction to practice of this finding, we think, that the combined inhibition of EGFR signaling and VEGFR signaling is superior to monotherapies against either against EGFR signaling or VEGFR signaling. Patients having tumors exhibiting elevated VEGFC expression and detectable EGFR do profit the most from treatments such as Sorafenib (BAY 43-9006), BAY 43-9005, BAY 57-9352, Sutent (SU11248), SU6668, Iressa, AZD6474, AZD2171, AZD6126, PTK787-ZK222584, CP547632, GW786034, CEP7055, Erbitux. Moreover patients, whose tumors in addition exhibit reduced or no expression of ERBB2, ERBB3 and ERBB4 have a particularly superior response to Sorafenib (BAY 43-9006), BAY 43-9005, BAY 57-9352, Sutent (SU11248), SU6668, Iressa, AZD6474, AZD2171, AZD6126, PTK787-ZK222584, CP547632, GW786034, CEP7055, Erbitux.

Therefore a method of any one of claims 1 to 3, comprising
(a) obtaining a biological sample from a patient;
(b) determining the pattern of expression levels of ERBB family member;
(c) comparing the pattern of expression levels determined in (b) with one or several reference pattern(s) of expression levels;
   wherein (i) downregulated expression of ERBB2, ERBB3 or ERBB4 is indicative of a poor prognosis as regards therapeutic success for said given mode of treatment in said subject is useful. In particular to select for anti VEGF-VEGFR regimens to treat the patients.

These conclusions and methods for diagnosis, prognosis and therapy guidance of anti cancer therapy management are relevant for all tumors potentially metastasizing via the lymphatic system. In particular this is important for lung, ovarian, cervix, stomach, pancreas, prostate, head and neck, renal cell, colon and breast cancer.

1. A method for predicting therapeutic success of a given mode of treatment in a patient having cancer or for adapting therapeutic regimen based on individualized risk assessment for a patient having cancer, comprising
(a) obtaining a biological sample from said patient;
(b) determining the pattern of expression levels of at least one marker gene of the group of marker genes listed in Table 1;
(c) comparing the pattern of expression levels determined in (b) with one or several reference pattern(s) of expression levels; and
(d) predicting therapeutic success for said given mode of treatment in said subject or implementing therapeutic regimen targeting said marker genes in said subject from the outcome of the comparison in step (c).

2. The method of claim 1, wherein in step (b) the pattern of expression levels of at least three marker genes is determined.

3. The method of claim 2, wherein in step (b) the pattern of expression levels of at least six marker genes is determined.

4. The method of claim 1, comprising
(a) obtaining a biological sample from a patient;
(b) determining at least the pattern of expression levels of VEGFC, ERBB3 and/or Her2/neu;
(c) comparing the pattern of expression levels determined in (b) with one or several reference pattern(s) of expression levels;
wherein (i) upregulated expression of VEGFC and/or (ii) downregulated expression of ERBB3 and/or Her2/neu is indicative of a poor prognosis as regards therapeutic success for said given mode of treatment in said subject.

5. The method of claim 1, wherein said given mode of treatment (a) acts on recruitment of lymphatic vessels, cell proliferation, cell survival and/or cell motility, and/or (b) comprises administration of a chemotherapeutic agent.

6. The method of claim 5, wherein said given mode of treatment comprises chemotherapy, administration of small molecule inhibitors, antibody based regimen, anti-proliferation regimen, pro-apoptotic regimen, pro-differentiation regimen, radiation and/or surgical therapy.

7. A method of selecting a therapy modality for a patient afflicted with a neoplastic disease, comprising
(a) obtaining a biological sample from said patient;
(b) predicting from said sample, by the method of any one of claims 1 to 6, therapeutic success for a plurality of individual modes of treatment; and
(c) selecting a mode of treatment which is predicted to be successful in step (b).

8. The method of claim 7, comprising
(a) obtaining a sample comprising cancer cells from said patient;
(b) separately maintaining aliquots of the sample in the presence of one or more test compositions;
(c) comparing expression of a single or plurality of marker genes, selected from the marker genes listed in Table 1 in each of the aliquots; and
(d) selecting a test composition which induces a lower level of expression of genes from Table 1 and/or a higher level of expression of genes from Table 1 in the aliquot containing that test composition, relative to the level of expression of each marker gene in the aliquots containing the other test compositions.

9. The method of claim 1, wherein the expression level is determined by
(a) a hybridization based method;
(b) real time real time PCR; or
(c) determining the protein level.

10. The method of claim 9, wherein said hybridization based method utilizes arrayed probes or individually labeled probes.

11. The method of claim 1, wherein said cancer or neoplastic disease is HNSCC, breast or colon cancer.

12. A kit useful for carrying out a method for predicting therapeutic success of a given mode of treatment in a patient having cancer or for adapting therapeutic regimen based on individualized risk assessment for a patient having cancer, comprising at least (a₁) three primer pairs and/or (a₂) three probes each having a sequence sufficiently complementary to the gene encoding VEGFC, ERBB3 and/or Her2/neu and/or (b) at least three antibodies directed against VEGFC, ERBB3 and Her2/neu.

13. A method for the treatment of a cancer associated with the recruitment of lymphatic vessels by expression of VEGFC comprising administering an effective amount of (a) an anti-VEGFC antibody, (b) an antisense nucleic acid or a ribozyme inhibiting the expression of the VEGFC encoding gene or (c) an inactive version of VEGFC as an antagonist.

14. The method according to claim 13, wherein said cancer is HNSCC, breast or colon cancer.

15. The method of claim 1, wherein in step (b) the pattern of expression levels of at one marker genes is determined.

16. The method of claim 1, wherein in step (b) the expression levels of VEGFC is determined.

17. The method of claim 1, wherein in step (b) the expression levels of ERBB2 is determined.

18. The method of claim 1, wherein in step (b) the expression levels of ERBB3 is determined.
